(19) United States
(12) Patent Application Publication
(10) Pub. No.: US 2026/0209309 A1
(43) Pub. Date: Jul. 23, 2026

(54) METHODS FOR MACROPHAGE POLARIZATION

(71) Applicant: UNIVERSITÄT ZÜRICH, Zürich (CH)

(72) Inventors: Simon BREDL, Zürich (CH); Roberto SPECK, Zürich (CH)

(73) Assignee: UNIVERSITÄT ZÜRICH, Zürich (CH)

(21) Appl. No.: 19/142,204

(22) PCT Filed: Dec. 20, 2023
(86) PCT No.: PCT/EP2023/087043

§ 371 Date: Jun. 22, 2025

(30) Foreign Application Priority Data
Dec. 21, 2022 (EP) 22215498.1
Sep. 29, 2023 (EP) 23200945.6

(51) Int. Cl. C07K 14/715; A61K 40/17; A61K 40/31; A61K 40/42; A61P 35/00; C07K 14/71; C07K 16/28; C12N 5/0786; C12N 15/86

(52) U.S. Cl. CPC C07K 14/7155; A61K 40/17; A61K 40/31; A61K 40/4204; A61K 40/4211; A61P 35/00; C07K 14/71; C07K 14/7156; C07K 16/2803; C07K 16/2863; C12N 5/0645; C12N 15/86; C07K 2317/622; C07K 2319/02; C12N 2510/00; C12N 2740/15043

(57) ABSTRACT

The present invention relates to a chimeric cytokine receptor and its use in switching the polarisation of macrophages or monocytes from M2 to M1. The present invention also relates to a method for monocyte modification to selectively increase the phagocytotic activity and to modulate the polarisation of offspring macrophages.

This application claims the right of priority of European Patent Applications EP22215498.1 filed 21 Dec. 2022, and EP23200945.6 filed 29 Sep. 2023, both of which are incorporated by reference herein.

### FIELD

The present invention relates to a chimeric cytokine receptor and its use in altering the polarisation of macrophages or monocytes from M2 to M1. The present invention also relates to a method for monocyte modification to selectively increase the phagocytotic activity and to modulate the polarisation of offspring macrophages.

### BACKGROUND

Monocytes are leukocytes circulating in the bloodstream that can differentiate inter alia to macrophages upon entering their target tissue. One of the main properties of macrophages is to engulf dead or diseased cells, which makes them an important part of the immune system that is relevant to numerous diseases.

Macrophages can adapt their immunological function depending on the activation signals they are exposed to in their target tissue. This process of macrophage polarization can give rise to M1 and M2 polarized macrophages. M1 macrophages, also known as classically activated macrophages, are important during acute infectious diseases, in particular infections by intracellular bacteria and viruses, but also for phagocytosis of tumor cells. M2 macrophages, also known as alternatively activated macrophages, are important for parasite defense and tissue remodeling.

In addition to the activation of macrophages, other mechanisms control their immune function. Engulfment of cells by macrophages is controlled by the CD47-SIRPα axis. Ligation of the ubiquitously expressed cell surface protein CD47 with the cell surface molecule SIRPα on macrophages transmits a “do-not-eat-me-signal” to the macrophages. This mechanism, which is meant to prevent macrophages from engulfing healthy cells, also prevents or at least decreases the effectiveness of macrophages in combating diseased cells. Some diseased cells, such as tumor cells, were found to overexpress CD47, thereby protecting themselves from macrophage phagocytosis.

Based on the above-mentioned state of the art, the objective of the present invention is to provide means and methods to improve macrophage activity against diseased cells. This objective is attained by the subject-matter of the independent claims of the present specification, with further advantageous embodiments described in the dependent claims, examples, figures and general description of this specification.

### SUMMARY OF THE INVENTION

A first aspect of the invention relates to an isolated monocyte or macrophage comprising a chimeric cytokine receptor (ChCR) polypeptide heterodimer comprising or consisting of a first ChCR polypeptide and a second ChCR polypeptide;
wherein the first ChCR polypeptide comprises:
   a first extracellular domain,
   a first type 1 transmembrane domain,
      optionally, a first flexible linker domain, linking said first extracellular domain and said first transmembrane domain,
   a first intracellular domain,
and wherein the second ChCR polypeptide comprises:
   a second extracellular domain,
   a second type 1 transmembrane domain,
      optionally, a second flexible linker domain, linking said second extracellular domain and said second transmembrane domain,
   a second intracellular domain,
wherein
   the first extracellular domain and the second extracellular domain are able to induce dimerization of said ChCR polypeptide heterodimer upon binding to a cytokine selected from IL-10 and TGFβ; and
   the first intracellular domain and the second intracellular domain are able to activate IFNγ receptor/Jak1/Jak2/STAT1 signalling in monocytes and/or macrophages upon dimerization of said ChCR polypeptide heterodimer. Said ChCR is inducing intracellular signalling (comparable to Interferon gamma stimulation) upon binding to IL-10 or TGFβ

A further aspect of the invention relates to an isolated monocyte or macrophage according to any one of the preceding aspects for use in treatment or prevention of cancer.

A further aspect of the invention relates to a kit comprising
an expression vector encoding a ChCR polypeptide as described above;
an inhibitory nucleic acid molecule directed against SIRPα as described above; and
an expression vector encoding a CAR polypeptide as described above.

A further aspect of the invention relates to a method for monocyte modification, comprising:
i. providing a monocyte obtained from a mammalian donor,
ii. inserting into said monocyte a nucleic acid sequence encoding a ChCR polypeptide according to the first aspect;
iii. maintaining said monocyte under cell culture conditions.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising”, “having”, “containing”, and “including”, and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a”, “or” and “the” include plural referents unless the context clearly dictates otherwise.

“And/or” where used herein is to be taken as specific recitation of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art (e.g., in cell culture, molecular genetics, nucleic acid chemistry, hybridization techniques and biochemistry, organic synthesis). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

Any patent document cited herein shall be deemed incorporated by reference herein in its entirety.

### Sequences

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

Reference to identical sequences without specification of a percentage value implies 100% identical sequences (i.e. the same sequence).

### General Biochemistry: Peptides, Amino Acid Sequences

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

The term peptide in the context of the present specification relates to a molecule consisting of up to 50 amino acids, in particular 8 to 30 amino acids, more particularly 8 to 15 amino acids, that form a linear chain wherein the amino acids are connected by peptide bonds.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (lie, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

In the context of the present specification, the term dimer refers to a unit consisting of two subunits.

In the context of the present specification, the term homodimer refers to a dimer comprised of two subunits that are either identical or are highly similar members of the same class of subunits.

In the context of the present specification, the term amino acid linker refers to a polypeptide of variable length that is used to connect two polypeptides in order to generate a single chain polypeptide. Exemplary embodiments of linkers useful for practicing the invention specified herein are oligopeptide chains consisting of 1, 2, 3, 4, 5, 10, 20, 30, 40 or 50 amino acids. A glycine-serine linker is composed of glycine and serine, while a glycine linker is composed of glycine subunits. A non-limiting example of an amino acid linker is the polypeptide GSGGGGSGGGGS (SEQ ID NO 023) that links an extracellular antigen binding domain with a transmembrane domain.

### General Molecular Biology: Nucleic Acid Sequences, Expression

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The term transgene in the context of the present specification relates to a gene or genetic material that has been transferred from one organism to another. In the present context, the term may also refer to transfer of the natural or physiologically intact variant of a genetic sequence into tissue of a patient where it is missing. It may further refer to transfer of a natural encoded sequence the expression of which is driven by a promoter absent or silenced in the targeted tissue.

The term recombinant in the context of the present specification relates to a nucleic acid, which is the product of one or several steps of cloning, restriction and/or ligation and which is different from the naturally occurring nucleic acid. A recombinant virus particle comprises a recombinant nucleic acid.

The terms gene expression or expression, or alternatively the term gene product, may refer to either of, or both of, the processes—and products thereof—of generation of nucleic acids (RNA) or the generation of a peptide or polypeptide, also referred to transcription and translation, respectively, or any of the intermediate processes that regulate the processing of genetic information to yield polypeptide products. The term gene expression may also be applied to the transcription and processing of a RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product.

The term knockdown of a mRNA in the context of the present specification relates to a reduction in the amount of this specific mRNA.

The term Nucleotides in the context of the present specification relates to nucleic acid or nucleic acid analogue building blocks, oligomers of which are capable of forming selective hybrids with RNA or DNA oligomers on the basis of base pairing. The term nucleotides in this context includes the classic ribonucleotide building blocks adenosine, guanosine, uridine (and ribosylthymine), cytidine, the classic deoxyribonucleotides deoxyadenosine, deoxyguanosine, thymidine, deoxyuridine and deoxycytidine. It further includes analogues of nucleic acids such as phosphothioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). Wherever reference is made herein to a hybridizing sequence, such hybridizing sequence may be composed of any of the above nucleotides, or mixtures thereof.

The term phosphothioate as used herein is synonymous with the terms phosphorothioate and thiophosphate.

The terms capable of forming a hybrid or hybridizing sequence in the context of the present specification relate to sequences that under the conditions existing within the cytosol of a mammalian cell, are able to bind selectively to their target sequence. Such hybridizing sequences may be contiguously reverse-complimentary to the target sequence, or may comprise gaps, mismatches or additional non-matching nucleotides. The minimal length for a sequence to be capable of forming a hybrid depends on its composition, with C or G nucleotides contributing more to the energy of binding than A or T/U nucleotides, and on the backbone chemistry.

In the context of the present specification, the term hybridizing sequence encompasses a polynucleotide sequence comprising or essentially consisting of RNA (ribonucleotides), DNA (deoxyribonucleotides), phosphothioate deoxyribonucleotides, 2′-O-methyl-modified phosphothioate ribonucleotides, LNA and/or PNA nucleotide analogues. In certain embodiments, a hybridizing sequence according to the invention comprises 8, 9, 10, 11, 12, 13, 14, 15, 16, 17, 18, 19, 20, 21, 22, 23, 24, 25, 26, 27, 28, 29 or 30 nucleotides. In certain embodiments, the hybridizing sequence is at least 80% identical, more preferred 85%, 90%, 92%, 94%, 95%, 96%, 97%, 98% or 99% identical to the reverse complimentary sequence of SEQ ID 33 to SEQ ID 42. In certain embodiments, the hybridizing sequence comprises deoxynucleotides, phosphothioate deoxynucleotides, LNA and/or PNA nucleotides or mixtures thereof.

In the context of the present specification, the term inhibitory nucleic acid molecule relates to a nucleic acid molecule which reduces the amount of functional mRNA of a target molecule inside the cell. Particularly, an inhibitory nucleic acid molecule relates to an oligonucleotide having a sequence substantially complimentary to, and capable of hybridizing to, an RNA. Antisense action on such RNA will lead to modulation, particular inhibition or suppression of the RNA's biological effect. If the RNA is an mRNA, expression of the resulting gene product is inhibited or suppressed. Inhibitory nucleic acid molecules can consist of DNA, RNA, nucleotide analogues and/or mixtures thereof. The skilled person is aware of a variety of commercial and non-commercial sources for computation of a theoretically optimal antisense sequence to a given target. Optimization can be performed both in terms of nucleobase sequence and in terms of backbone (ribo, deoxyribo, analogue) composition. Many sources exist for delivery of the actual physical oligonucleotide.

The term antisense oligonucleotide in the context of the present specification relates to an oligonucleotide having a sequence substantially complimentary to, and capable of hybridizing to, an RNA. Antisense action on such RNA will lead to modulation, particular inhibition or suppression of the RNA's biological effect. If the RNA is an mRNA, expression of the resulting gene product is inhibited or suppressed. Antisense oligonucleotides can consist of DNA, RNA, nucleotide analogues and/or mixtures thereof. The skilled person is aware of a variety of commercial and non-commercial sources for computation of a theoretically optimal antisense sequence to a given target. Optimization can be performed both in terms of nucleobase sequence and in terms of backbone (ribo, deoxyribo, analogue) composition. Many sources exist for delivery of the actual physical oligonucleotide, which generally is synthesized by solid state synthesis.

The term siRNA (small/short interfering RNA) in the context of the present specification relates to an RNA molecule capable of interfering with the expression (in other words: inhibiting or preventing the expression) of a gene comprising a nucleic acid sequence complementary or hybridizing to the sequence of the siRNA in a process termed RNA interference. The term siRNA is meant to encompass both single stranded siRNA and double stranded siRNA. siRNA is usually characterized by a length of 17-24 nucleotides. Double stranded siRNA can be derived from longer double stranded RNA molecules (dsRNA). According to prevailing theory, the longer dsRNA is cleaved by an endo-ribonuclease (called Dicer) to form double stranded siRNA. In a nucleoprotein complex (called RISC), the double stranded siRNA is unwound to form single stranded siRNA. RNA interference often works via binding of an siRNA molecule to the mRNA molecule having a complementary sequence, resulting in degradation of the mRNA. RNA interference is also possible by binding of an siRNA molecule to an intronic sequence of a pre-mRNA (an immature, non-spliced mRNA) within the nucleus of a cell, resulting in degradation of the pre-mRNA.

The term shRNA (small hairpin RNA) in the context of the present specification relates to an artificial RNA molecule with a tight hairpin turn that can be used to silence target gene expression via RNA interference (RNAi).

The term sgRNA (single guide RNA) in the context of the present specification relates to an RNA molecule capable of sequence-specific repression of gene expression via the CRISPR (clustered regularly interspaced short palindromic repeats) mechanism.

The term miRNA (microRNA) in the context of the present specification relates to a small non-coding RNA molecule (containing about 22 nucleotides) that functions in RNA silencing and post-transcriptional regulation of gene expression.

The term nucleic acid expression vector in the context of the present specification relates to an artificial nucleic acid molecule used as a vehicle to carry foreign nucleic acid molecules into another cell. The nucleic acid expression vector relates to a plasmid, a viral genome or an RNA, which is used to transfect (in case of a plasmid or an RNA) or transduce (in case of a viral genome) a target cell with a certain gene of interest, or—in the case of an RNA construct being transfected—to translate the corresponding protein of interest from a transfected mRNA. For vectors operating on the level of transcription and subsequent translation, the gene of interest is under control of a promoter sequence and the promoter sequence is operational inside the target cell, thus, the gene of interest is transcribed either constitutively or in response to a stimulus or dependent on the cell's status. In certain embodiments, the viral genome is packaged into a capsid to become a viral vector, which is able to transduce the target cell.

The term promoter in the context of the present specification relates to a nucleic acid sequence that initiates the transcription of a particular nucleic acid sequence in a macrophage. Different promoters are well known in the art and they are used widely in genetics as part of vectors that contains nucleic acid sequences to be transcribed. A specific type of promoters is only active under certain conditions either in the presence or absence of certain molecules (inducible promoters) or in a certain cellular environment such as cell type specific promoters.

The term chimeric antigen receptor in the context of the present specification relates to artificial engineered receptors comprising parts of antigen receptors.

An immunoreceptor tyrosine-based activation motif (ITAM) is an important component of the intracellular signaling machinery of cell surface proteins of the immune system. The ITAM is located on the cytoplasmic tail of the cell surface protein and becomes phosphorylated on its tyrosine residue after interaction of the cell surface protein with its respective ligand. The phosphorylated tyrosine residue forms a docking site for other downstream components in the signaling machinery. The ITAM motif comprises a tyrosine separated from a leucine or isoleucine by any other two amino acids.

The term chimeric cytokine receptor in the context of the present specification relates to artificial engineered receptors comprising parts of cytokine receptors.

Type II cytokine receptors are transmembrane proteins that are expressed on the surface of certain cells, which bind and respond to a select group of cytokines. These receptors are similar to type I cytokine receptors except that they do not possess the signature sequence WSXWS which is characteristic of type I receptors. Typically type II cytokine receptors are heterodimers or multimers with a high and a low affinity component.

Type I cytokine receptors are transmembrane receptors expressed on the surface of cells that recognize and respond to cytokines with four α-helical strands. These receptors are also known under the name hemopoietin receptors, and share a common amino acid motif (WSXWS) in the extracellular portion adjacent to the cell membrane. Members of the type I cytokine receptor family comprise different chains, some of which are involved in ligand/cytokine interaction and others that are involved in signal transduction.

A type 1 transmembrane domain is derived from a type I membrane protein.

The term type 1 membrane protein in the context of the present specification relates to single-pass membrane proteins anchored to the lipid membrane with their N-terminal domain targeted to the ER lumen during synthesis and the extracellular space in their mature form.

The term IFNγ receptor/Jak1/Jak2/STAT1 signalling in monocytes and/or macrophages in the context of the present specification relates to proinflammatory activation of monocytes/macrophages via the Interferon γ receptor signaling cascade. The Interferon γ receptor-induced Jak1/Jak2/STAT1 signaling switches monocyte differentiation form dendritic cells to macrophages and induces the production of proinflammatory cytokines in macrophages turning them to proinflammatory “M1” macrophages. The interferon γ receptor signaling may be initiated by the ChCR of the invention. The M1 activation of macrophages are positively associated with longer survival times and most positive clinical outcomes in many cancers.

### (Cancer) Immunotherapy

In the context of the present specification, the term cancer immunotherapy, biological or immunomodulatory therapy is meant to encompass types of cancer treatment that help the immune system to fight cancer.

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

The term cancer as used in the context of the present specification relates to malignant neoplastic disease; the terms “cancer” and “malignant neoplastic disease” are used synonymously herein. They specifically include carcinoma (epithelial derived cancer), sarcoma (connective tissue derived cancer), lymphoma and leukemia, germ-cell derived tumours and blastomas. Particular alternatives of any of the aspects and embodiments disclosed herein are directed at the use of the compounds and compositions of the invention in treatment of solid tumours. Other alternatives of any of the aspects and embodiments disclosed herein are directed at the use of the combinations of the invention in treatment of liquid cancers such as myelogenous or granulocytic leukemia, particularly AML, lymphatic, lymphocytic, or lymphoblastic leukemia and lymphoma, polycythemia vera or erythremia.

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

### DETAILED DESCRIPTION OF THE INVENTION

A first aspect of the invention relates to an isolated monocyte or macrophage cell. The isolated monocyte or macrophage cell comprises a chimeric cytokine receptor (ChCR) polypeptide heterodimer. The ChCR polypeptide heterodimer comprises or consists of a first ChCR polypeptide and a second ChCR polypeptide.

The first ChCR polypeptide comprises, particularly from N to C terminus:
a first extracellular domain,
a first type 1 transmembrane domain,
   optionally, a first flexible linker domain, linking said first extracellular domain and said first transmembrane domain,
a first intracellular domain.

The second ChCR polypeptide comprises, particularly from N to C terminus:
a second extracellular domain,
a second type 1 transmembrane domain,
   optionally, a second flexible linker domain, linking said second extracellular domain and said second transmembrane domain,
a second intracellular domain.

The first extracellular domain and the second extracellular domain are able to induce dimerization of said ChCR polypeptide heterodimer upon binding to a cytokine selected from IL-10 and TGFβ.

The first intracellular domain and the second intracellular domain are able to activate IFNγ receptor/Jak1/Jak2/STAT1 signalling in monocytes and/or macrophages upon dimerization of said ChCR polypeptide heterodimer.

In certain embodiments,
the first extracellular domain is or comprises an IL-10 receptor α (CDW210A) extracellular domain;
the second extracellular domain is or comprises an IL-10 receptor β (CDW210B) extracellular domain;
the first intracellular domain is or comprises an IFNγ receptor 1 (CD119) intracellular domain; and
the second intracellular domain is or comprises an IFNγ receptor 2 intracellular domain.

In certain embodiments, the ChCR polypeptide heterodimer comprises:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 21,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 22,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
   particularly wherein said ChCR polypeptide heterodimer has a biological activity of ≥85%, particularly ≥90%, ≥95% of a ChCR heterodimer of the sequences SEQ ID NO 53 and SEQ ID NO 54.

In certain embodiments,
the first extracellular domain is or comprises an IL-10 receptor α (CDW210A) extracellular domain;
the second extracellular domain is or comprises an IL-10 receptor β (CDW210B) extracellular domain;
the first intracellular domain is or comprises an IFNγ receptor 2 intracellular domain; and
the second intracellular domain is or comprises an IFNγ receptor 1 (CD119) intracellular domain.

In certain embodiments, the ChCR polypeptide heterodimer comprises:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 21,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 22,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
particularly wherein said ChCR polypeptide heterodimer has a biological activity of ≥85%, particularly ≥90%, ≥95% of a ChCR heterodimer of the sequences SEQ ID NO 59 and SEQ ID NO 60.

In certain embodiments,
the first extracellular domain is or comprises a TGFβ receptor type I extracellular domain;
the second extracellular domain is or comprises a TGFβ receptor type II extracellular domain;
the first intracellular domain is or comprises an IFNγ receptor 1 (CD119) intracellular domain; and
the second intracellular domain is or comprises an IFNγ receptor 2 intracellular domain.

In certain embodiments, the ChCR polypeptide heterodimer comprises:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 23,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 24,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
particularly wherein said ChCR polypeptide heterodimer has a biological activity of ≥85%, particularly ≥90%, ≥95% of a ChCR heterodimer of the sequences SEQ ID NO 55 and SEQ ID NO 56.

In certain embodiments,
the first extracellular domain is or comprises a TGFβ receptor type I extracellular domain;
the second extracellular domain is or comprises a TGFβ receptor type II extracellular domain;
the first intracellular domain is or comprises an IFNγ receptor 2 intracellular domain; and
the second intracellular domain is or comprises an IFNγ receptor 1 (CD119) intracellular domain.

In certain embodiments, the ChCR polypeptide heterodimer comprises:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 23,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 24,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
particularly wherein said ChCR polypeptide heterodimer has a biological activity of ≥85%, particularly ≥90%, ≥95% of a ChCR heterodimer of the sequences SEQ ID NO 61 and SEQ ID NO 62.

The biological activity of the chimeric cytokine receptor can be measured in reporter cells which are HEK-Blue™ IFN-γ cells (InvivoGen). Briefly, the ChCR is introduced into the HEK-Blue™ IFN-γ cell line, which contains an IFN-γ driven STAT-1 inducible secreted alkaline phosphatase (SEAP) reporter but lacks any IL-10 receptor or TGFβ receptor, respectively. The amount of SEAP can be easily assessed in a colorimetric assay (QuantiBlue) and the SEAP signal is proportional to the biological activity.

A further aspect of the invention relates to an isolated monocyte or macrophage comprising a nucleic acid molecule encoding the ChCR polypeptide as described in the first aspect. The nucleic acid molecule encodes the ChCR together with a signal peptide. The signal peptide effects transport to the cell surface of the ChCR.

In certain embodiments, the nucleic acid molecule is comprised in an expression vector, wherein the ChCR polypeptide heterodimer is under control of a promoter sequence operable in a mammalian monocyte or macrophage. In certain embodiments, the expression vector is selected from the group of a viral vector, a plasmid, a DNA molecule, or an RNA molecule. In certain embodiments, the expression vector is a lentiviral vector.

In certain embodiments, the monocyte or macrophage additionally comprises an inhibitory nucleic acid molecule directed against SIRPα.

In certain embodiments, the inhibitory nucleic acid molecule directed against SIRPα comprises a sequence selected from the group of SEQ ID NO 33 to SEQ ID NO 52.

In certain embodiments, the monocyte or macrophage additionally comprises a chimeric antigen receptor (CAR) polypeptide, comprising:
an extracellular antigen binding domain,
a transmembrane domain, and
an intracellular domain comprising at least one ITAM motif.

In certain embodiments, the CAR comprises a linker domain, linking said extracellular antigen binding domain and said transmembrane domain.

In certain embodiments, the monocyte or macrophage additionally comprises a chimeric antigen receptor (CAR) polypeptide, comprising:
an extracellular antigen binding domain comprising a Fab-fragment,
a transmembrane domain comprising a CD8 transmembrane domain, and
an intracellular domain comprising the intracellular domain of a FC-receptor.

In certain embodiments, the intracellular domain of the CAR comprises the intracellular domain of a FCgamma-receptor. In certain embodiments, the intracellular domain of the CAR comprises the intracellular domain of CD32a.

In certain embodiments, the CAR polypeptide comprises a sequence having at least ≥80%, ≥85%, ≥90%, ≥92%, ≥94%, ≥95%, ≥96%, ≥97%, ≥98%, ≥99%, or 100% identity to any one of SEQ ID NO 13 to SEQ ID NO 18.

A further aspect of the invention relates to an isolated monocyte or macrophage according to the fourth aspect for use in medicine.

A further aspect of the invention relates to an isolated monocyte or macrophage according to any one of the preceding aspects for use in treatment or prevention of cancer.

A further aspect of the invention relates to a kit comprising
an expression vector encoding a ChCR polypeptide as described above;
an inhibitory nucleic acid molecule directed against SIRPα as described above;
and
an expression vector encoding a CAR polypeptide as described above.

In certain embodiments, the components of the kit are encoded on a single vector, for example on a lentiviral vector. In certain embodiments, the components of the kit are encoded on two or three vectors.

A further aspect of the invention relates to a method for monocyte modification, comprising:
i. providing a monocyte obtained from a mammalian donor,
ii. inserting into said monocyte a nucleic acid sequence encoding a ChCR polypeptide as described above;
iii. maintaining said monocyte under cell culture conditions.

In certain embodiments of the method, additionally,
an inhibitory nucleic acid molecule directed against SIRPα as described above; and/or
a CAR polypeptide as described above;
   is inserted into said monocyte.

A further aspect of the invention relates to a ChCR polypeptide heterodimer comprising:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 21,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 22,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29.

A further aspect of the invention relates to a ChCR polypeptide heterodimer comprising:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 21,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 22,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28.

A further aspect of the invention relates to a ChCR polypeptide heterodimer comprising:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 23,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 24,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29.

A further aspect of the invention relates to a ChCR polypeptide heterodimer comprising:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 23,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 24,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28.

### Medical Treatment

Similarly, within the scope of the present invention is a method or treating cancer in a patient in need thereof, comprising administering to the patient a monocyte or macrophage according to the above description.

### Method of Manufacture and Method of Treatment According to the Invention

The invention further encompasses, as an additional aspect, the use of a monocyte or macrophage as identified herein, for use in a method of manufacture of a medicament for the treatment or prevention of cancer.

Similarly, the invention encompasses methods of treatment of a patient having been diagnosed with a disease associated with cancer. This method entails administering to the patient an effective amount of a monocyte or macrophage as identified herein.

Wherever alternatives for single separable features such as, for example, a monocyte or macrophage or a receptor sequence or a medical indication are laid out herein as “embodiments”, it is to be understood that such alternatives may be combined freely to form discrete embodiments of the invention disclosed herein. Thus, any of the alternative embodiments for a monocyte or macrophage may be combined with any of the alternative embodiments of receptor sequence and these combinations may be combined with any medical indication mentioned herein.

The specification further encompasses the following items:

### Items:

1. A CAR polypeptide comprising a sequence having at least ≥80%, ≥85%, ≥90%, ≥92%, ≥94%, ≥95%, ≥96%, ≥97%, ≥98%, ≥99%, or 100% identity to any one of SEQ ID NO 13 to SEQ ID NO 18.
2. A ChCR polypeptide heterodimer comprising:
   a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 21,
   a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
   a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
   a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 22,
   a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
   a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
wherein said ChCR polypeptide heterodimer has a biological activity of 85%, particularly ≥90%, ≥95% of a ChCR heterodimer of the sequences SEQ ID NO 53 and SEQ ID NO 54.
3. A ChCR polypeptide heterodimer comprising:
   a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 23,
   a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
   a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
   a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 24,
   a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
   a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
wherein said ChCR polypeptide heterodimer has a biological activity of 85%, particularly ≥90%, ≥95% of a ChCR heterodimer of the sequences SEQ ID NO 55 and SEQ ID NO 56.
4. A ChCR polypeptide heterodimer comprising:
   a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 21,
   a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
   a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
   a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 22,
   a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
   a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
wherein said ChCR polypeptide heterodimer has a biological activity of 85%, particularly ≥90%, ≥95% of a ChCR heterodimer of the sequences SEQ ID NO 59 and SEQ ID NO 60.
5. A ChCR polypeptide heterodimer comprising:
   a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 23,
   a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
   a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
   a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 24,
   a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
   a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
wherein said ChCR polypeptide heterodimer has a biological activity of 85%, particularly ≥90%, ≥95% of a ChCR heterodimer of the sequences SEQ ID NO 61 and SEQ ID NO 62.

The invention is further illustrated by the following examples and figures, from which further embodiments and advantages can be drawn. These examples are meant to illustrate the invention but not to limit its scope.

### DESCRIPTION OF THE FIGURES

FIG. 1 shows A) Knockdown of SIRPα in THP-1 cells with nine different siRNA against SIRPα. Protein expression was normalized to an unspecific «non-targeting» siRNA (siRNA NT). B) The inventors further characterized the three most promising SIRPα specific siRNAs (3, 6 and 9) as miRNAs in THP-1 by transducing them with a lentiviral vector expressing these miRNAs. C) The most promising miRNA 3 was then verified in primary human macrophages. As negative control, the inventors used an unspecific «non-targeting» miRNA (miRNA NT). SIRPα expression was measured by flow cytometry and the measured median fluorescent intensity (MFI) was normalized to miRNA NT.

FIG. 2 shows CD19 specific phagocytosis of tumor cells by mCAR expressing macrophages. A) Cartoon of the mCAR design: An extracellular human CD19 specific scFv antibody fragment is linked via a CD8a stalk and transmembrane domain to an intracellular signaling domain, which contain ITAMs. B) and C) The inventors assessed the phagocytic activity of the mCAR in transduced macrophages, which also express RFP, by FACS. As target served CFSE labelled Raji cells. Macrophages, which phagocytosed Raji cells, are RFP and CFSE double positive. The assay was made with macrophages from four different donors.

FIG. 3 shows EGFR specific phagocytosis of tumor cells by mCAR expressing macrophages. A) EGFR binding of Cetuximab mCAR expressing human primary macrophages. B) The inventors assessed the phagocytic activity of the Cetuximab mCAR in transduced macrophages, which also express RFP, by FACS. As targets served CFSE labelled MDA-MB-231 cells, which express EGFR. Macrophages, which phagocytosed MDA-MB-231 cells, are RFP and CFSE double positive. The assay was made with macrophages from two different donors. Macrophages transduced with the CD19 specific mCAR served as control for unspecific phagocytosis. In addition, both experimental groups were treated with Cytochalasin D (CytD) which inhibits phagocytosis.

FIG. 4 shows generation of a functional chimeric cytokine receptor (ChCR). A) Design and mode of action of the IL-10 ChCR. The extracellular part is binding to IL-10, whereas the intracellular domain derived from the IFN-γ receptor induces the STAT1 signaling pathway leading to a M1 polarization of the macrophage. B) The inventors assessed the function of the ChCR IL-10-IFNGR pairs in a cell line expressing the gene for SEAP driven by IFN-γ. Adding IL-10 to the cells transduced with both IL-10 ChCR subunits gave a positive signal. STAT1 phosphorylation upon IL-10 stimulation of the IL-10 ChCR was verified by Westernblot in C) the transduced reporter cells and in D) in transduced THP-1. The transduced THP-1 reacted in a dose dependent manner to the IL-10 stimulation. E) STAT1 phosphorylation was further verified by flow cytometry in primary macrophages either left untransduced, mock transduced or transduced with IL-10 ChCR.

FIG. 5 shows results of stimulating the gene engineered macrophages expressing the ChCR with IL-10. IL-10 ChCR expressing monocyte-derived macrophages (MDMs) are M1 polarized upon stimulation with IL-10. Monocytes from healthy donors were either left untransduced, mock transduced or transduced with IL-10 ChCR expressing lentiviruses. After differentiation to MDMs, they were polarized for 2d either with 5 ng/ml IFN-γ or 100 ng/ml IL-10. Expression of polarization markers was assessed by flow cytometry. N=3-4, p-values were calculated using paired t-tests (for % CD38 and CD163+) and ratio paired t-test (for MFIs)

FIG. 6 shows TGF-β ChCR induces pSTAT1. Flow cytometer analysis of STAT1 phosphorylation in THP-1 cells and macrophages either mock transduced or transduced with the TGF-β ChCR. Cells were stimulated as indicated and pSTAT1 measured by flow cytometry.

FIG. 7 shows results of stimulating the gene-engineered macrophages expressing the TGF-β ChCR with TGF-β. ChCR expressing monocyte-derived macrophages (MDMs) are M1 polarized upon stimulation with TGFβ. Monocytes from healthy donors were either mock transduced or transduced with TGF-β ChCR expressing lentiviruses. After differentiation to MDMs, they were polarized for 2 days as indicated. Expression of surface markers was assessed by flow cytometry. N=3, p-values were calculated using paired t-tests (for CD38+) and ratio paired t-test (for MFIs).

FIG. 8 shows IP-10 secretion of ChCR expressing monocyte-derived macrophages (MDMs) upon stimulation with TGF-β or IL-10. Monocytes from healthy donors were either left untransduced, mock transduced or transduced with ChCR expressing lentiviruses. After differentiation to MDMs, they were polarized for 2 days with either IFN-γ or IL-10 or TGFβ at indicated concentrations. IP-10 secretion in the supernatant was analyzed by ELISA. N=4.

FIG. 9 shows the tumoricidal effect of conditioned medium from gene engineered macrophages expressing a ChCR on TNBC cell lines. Monocytes from healthy donors were either left untransduced, mock transduced or transduced with IL-10 ChCR (A), or TGFβ ChCR (B) expressing lentiviruses. After differentiation to MDMs, they were polarized for 2 days as indicated. The conditioned medium from polarized macrophages was used to treat MDA-MB-231 or BT-549 cell line for 3 days. As a control, MDA-MB-231 and BT-549 were directly treated with cytokines used to stimulate macrophages. Viability was assessed using a WST1 assay. Shown are results using conditioned medium from 1-4 donors.

FIG. 10 shows A) the transduction efficacy of transductions of human primary macrophages with an empty lentiviral vector (“mock), with the IL-10 ChCR and the TGF-β ChCR expressing lentiviral vector. N=14-33 B) Surface expression on human primary macrophages of the two subunits of the IL-10 ChCR, IL-10Ra and IL-10Rb, displayed as MFI and % IL-10Ra and IL-10Rb+, N=4. C) Surface expression on human primary macrophages of the TGF-β ChCR displayed as MFI and % TGFbR2+ cells, N=5.

### EXAMPLES

### Example 1: SIRPα miRNA: Enhancing the Phagocytic Activity of Macrophages

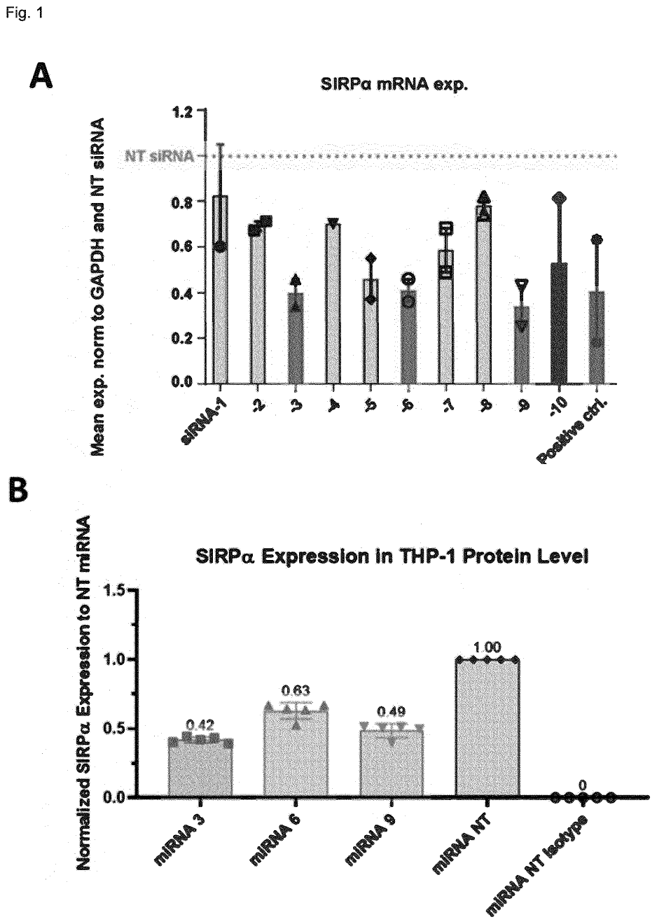

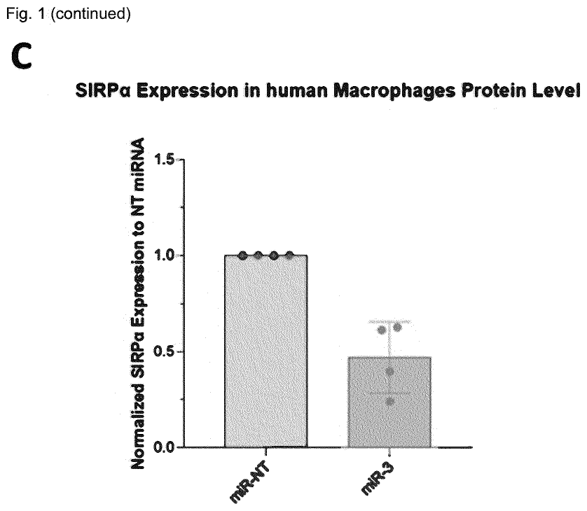

The signal regulatory protein (SIRP)-α on MΦ binds to the cell surface molecule CD47, which is ubiquitously present on rather all cells. Recognition of CD47 by SIRPα transmits an inhibitory “do not-eat me signal” to the MΦ. Thus, the SIRPα-CD47 axis acts as a master checkpoint for phagocytosis and is involved in the cell turnover of senescent cells. Notably, senescent cells lose their CD47 expression and thus are prone for phagocytosis. By virtue of the potential to harness the phagocytosis, this “do-not-eat me signal” has moved in the focus of novel cancer therapies. The inventors will promote the MΦs' phagocytic activity by down-regulating, SIRPα expression using specific microRNA (miRNA). miRNA are small non-coding RNA molecules, which regulate post-transcriptionally gene expression. The inventors identified the macrophage-like cell line, THP-1, which expresses SIRPα at high levels, and thus was optimal for screening the miRNA directed against SIRPα. In THP-1, the inventors screened in total 10 siRNA candidates (FIG. 1A) and verified three of them as miRNAs with different knockdown capacities (FIG. 1B). The inventors then transduced human monocytes isolated from peripheral blood mononuclear cells (PBMCs) with lentiviral vectors encoding the most promising one, miRNA3. The inventors verified then the SIRPα knockdown in primary human macrophages (FIG. 1C). A non-targeting (NT) miRNA served as negative control. Indeed, miRNA 3 downregulated the SIRPα expression in primary human macrophages to a level of about 0.5 as compared to the NT miRNA level of 1.0.

### Example 2: Generation of Chimeric Antigen Receptors (mCAR), which are Expressed on Macrophages and Induce Antigen Specific Phagocytosis of Tumor Cells

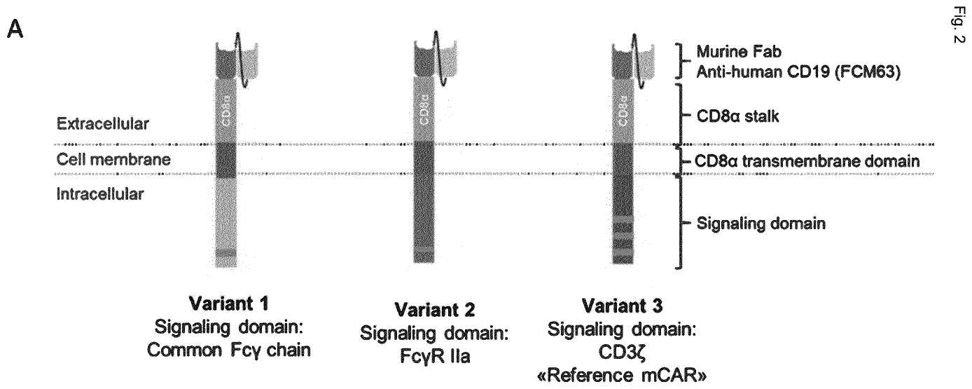

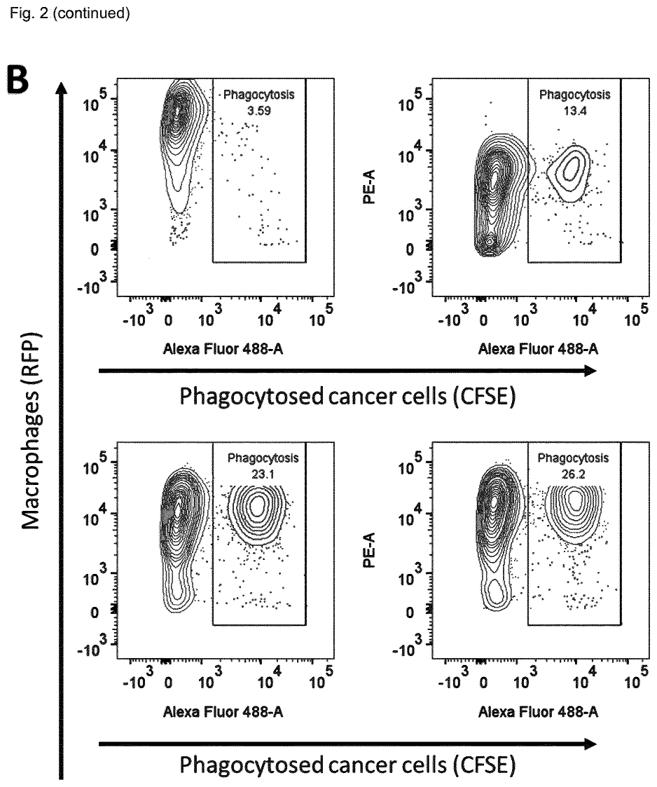

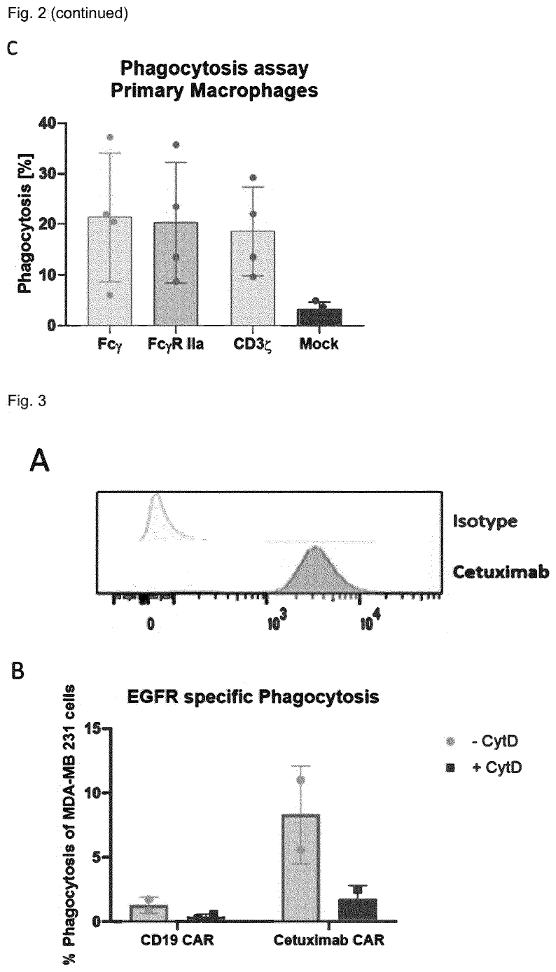

In a first instance, the inventors are investigating which cytoplasmic tail activates the genetically modified macrophages best. Thus, the inventors are working with an identical external part of the CAR in all constructs designed, i.e., the inventors designed a macrophage chimeric antigen receptor (mCAR) with a single chain variable fragment (scFv) murine antibody, i.e., FMC63, which binds to human CD19 (huCD19) and has successfully used for CAR T-cell receptors (FIG. 2A). Importantly, the amino acid sequence of the anti-CFD19 scFv is publically available (ADM64594.1), suitable reagents are commercially available to evaluate the mCAR, and CD19 expressing Raji cells, which serve as targets for the CAR expressing cells, were already available.

The cytoplasmic tails the inventors are exploring are the FcγR chain and the FcγRIIa (CD32A) (FIG. 2A Variant 1 and 2). As a reference, the inventors included the CAR published by the company CARISMA, which contains the CD3ζ signaling domain. The CD3ζ is normally part of the T-cell receptor complex but it seems that it can also induce Syk signaling in macrophages and thus promotes phagocytosis. Nevertheless, the inventors are convinced that the Fcγ and FcγRIIa chains, because they are inherently expressed by macrophages and play a major role in antibody dependent cell phagocytosis (ADCP), are the better choice for creating mCAR. The common feature of these FcγR chains are immunoreceptor tyrosine-based activation motifs (ITAMs) and an activation of those via phosphorylation results in vigorous Syk signaling in macrophages and promotes antigen-specific cellular phagocytosis.

The inventors found that all three mCAR showed similar cell surface expression levels and antigen binding capacity (data not shown). More intriguingly, the inventors verified the antigen specific phagocytosis by primary human macrophages following their recognition of CD19 expressing target cells (Raji cells). Briefly, the inventors transduced the macrophages with the viral vectors encoding the different receptors and then co-incubated them with fluorescently labeled Raji cells as target cells. The inventors also transduced macrophages with a lentiviral vector encoding solely the reporter gene but lacking the mCAR (“Mock” control). The inventors found that the macrophages with all the different mCAR phagocytosed substantially more Raji cells than the “mock” transduced ones (FIG. 2B)—there was no difference between the types of cytoplasmic tails (FIG. 2C).

We made then a first characterization in primary human macrophages of the EGFR specific Cetuximab mCAR for its antigen binding capacity. The inventors stained transduced macrophages with recombinant EGFR protein and verified the binding of the proteins by staining for the Avi-tag of the recombinant EGFR (FIG. 3A). The inventors also evaluated the Cetuximab mCAR for its ability to induce antigen specific phagocytosis of the EGFR expressing tumor cell line MDA-MB-231. As control severed the CD19 specific mCAR which should not induce phagocytosis since the MDA-MB-231 cell line does not express CD19. As further control, the inventors added Cytochalasin D, which inhibits phagocytosis, to show that the mCAR induces phagocytosis and not only binding of the target cells. The inventors can clearly show that the Cetuximab mCAR induces specifically phagocytosis of the MDA-MB-231 cells.

### Example 3: Macrophage Chimeric Cytokine Receptor

The TME is rich in anti-inflammatory cytokines such as IL-10 and TGF-β. In fact, the inventors will benefit from the presence of IL-10 or TGF-β in the TME. To do so, the inventors will generate genetically modified macrophages expressing chimeric cytokine receptor, which binds for example IL-10 but triggers a pro-inflammatory signal via the cytosolic IFN-γ chain.

### Example 4: The IL-10-IFNγ Chimeric Cytokine Receptor (IL-10 ChCR)

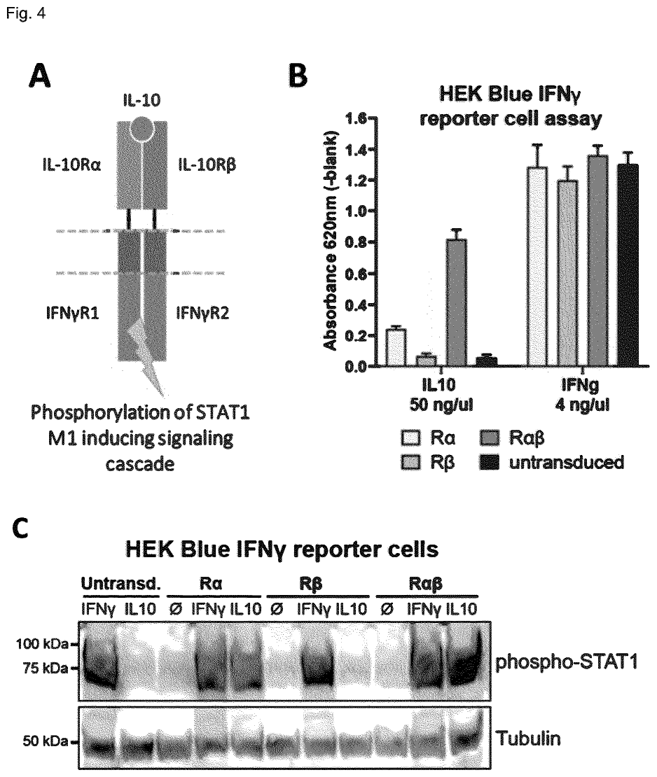

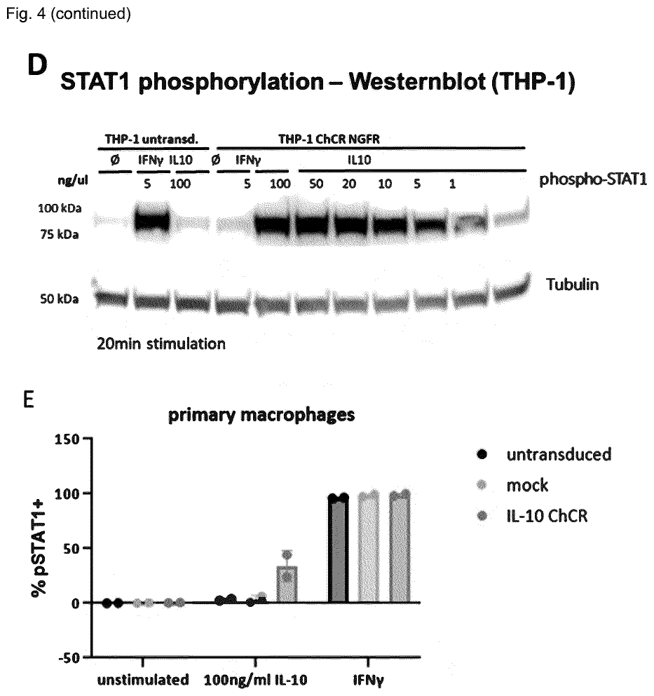

As first prototype, the inventors designed and generated a chimeric cytokine receptor pair with the extracellular recognition domains of IL-10, i.e., IL-10Rα and Rβ, and the intracellular cytoplasmic domains of IFN-γR1 and -R2 (FIG. 4A).

Homodimerization of the prototype chimeric cytokine receptor in response to IL-10 binding should trigger the IFN-γ signaling pathway via phosphorylation of the signal transducer and activator of transcription 1 (STAT1) by the corresponding cytosolic domains. PhosphoSTAT1 signaling triggered by IFN-γ results in the polarization of MCD towards the classical “M1” instead of the alternative “M2” phenotype.

The inventors verified the expression and the functionality of the chimeric cytokine receptor in HEK-Blue™ IFN-γ cells (InvivoGen) (FIG. 4B). Briefly, the inventors introduced the IL-10 ChCR into the HEK-Blue™ IFN-γ cell line, which contains an IFN-γ driven STAT-1 inducible secreted alkaline phosphatase (SEAP) reporter but lacks any IL-10 receptor. The amount of SEAP can be easily assessed in a colorimetric assay (QuantiBlue). The expression of IL-10Rα-IFN-γR1 together with IL-10Rβ-IFN-γR2 in response to IL-10 resulted in the highest SEAP activity overall. In addition, the inventors observed increased SEAP activity in IL-10 stimulated cells transduced solely with Ra but not with Rβ. The inventors concluded that high expression of the high affinity IL-10Rα-IFN-γR1 subunit suffices upon binding to IL-10 to induce some STAT1 signaling. The inventors corroborated the data above by demonstrating STAT1 phosphorylation upon ChCR stimulation with IL-10 via Westernblot in HEK-Blue™ IFN-γ cells and in the human monocytic cell line THP-1 (FIGS. 4C and 4D). Also in primary human macrophages, the inventors show STAT1 phosphorylation upon ChCR stimulation with IL-10 (FIG. 4E). STAT1 is the first non-receptor kinase phosphorylated upon activating certain cytokine receptors. Here, the inventors induce the IFNγ signaling pathway by stimulating the chimeric cytokine with IL-10.

Achieving this data with the IL-10 ChCR, the inventors started with the generation of a second variant of the ChCR consisting of the extracellular domains of the TGF-β receptor and the intracellular domains of IFN-γ receptor (TGFβ chimeric cytokine receptor).

As a next step, the inventors transduced primary human monocytes with the IL-10 ChCR lentiviral vector and as control served monocytes transduced with a control lentivirus (“mock”) or were left untransduced. After transduction, the inventors differentiated the monocytes to macrophages. The macrophages were either stimulated with IFNγ for M1 activation, with IL-10 for M2 activation or were left unstimulated. After stimulation, the inventors harvested the cells and analyzed the expression of HLA-DR and CD38 as marker for M1 activation and CD163 as a marker for M2 activation. (FIG. 5)

### Example 5: The TGF-β-IFNγ Chimeric Cytokine Receptor (TGF-β ChCR)

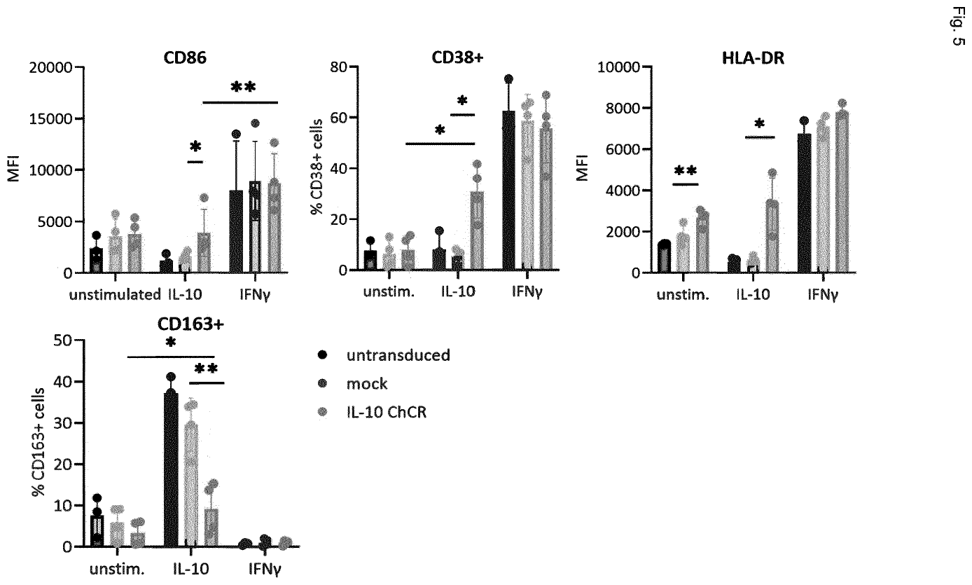

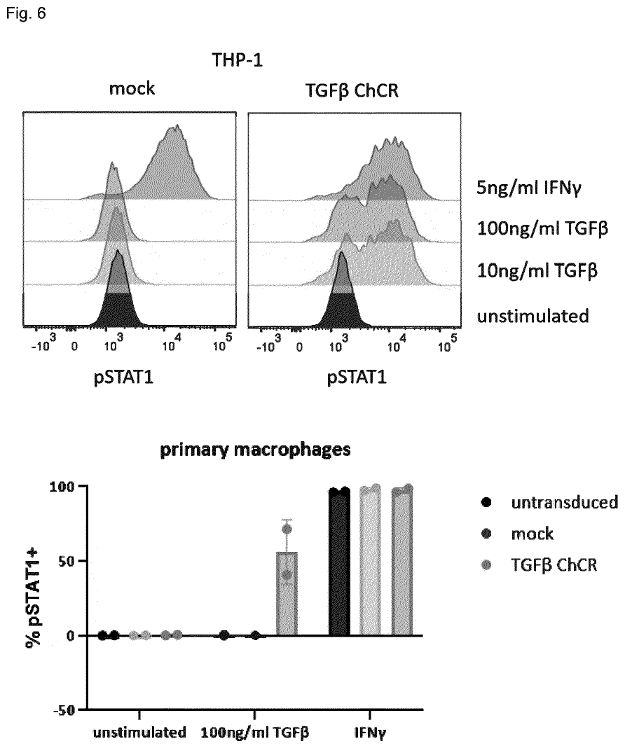

As mentioned above, after obtaining first functional data on the prototype ChCR, the inventors started with TGF-β-IFNγ ChCR combination. The inventors already gathered first data on this ChCR variant in THP-1 and human primary macrophages. TGF-β strongly induces pSTAT1 solely in THP-1 cells and human primary macrophages expressing the TGF-β ChCR. THP-1 cells and human primary macrophages transduced with an irrelevant lentiviral vector (“Mock”) did not phosphorylate STAT1 (FIG. 6).

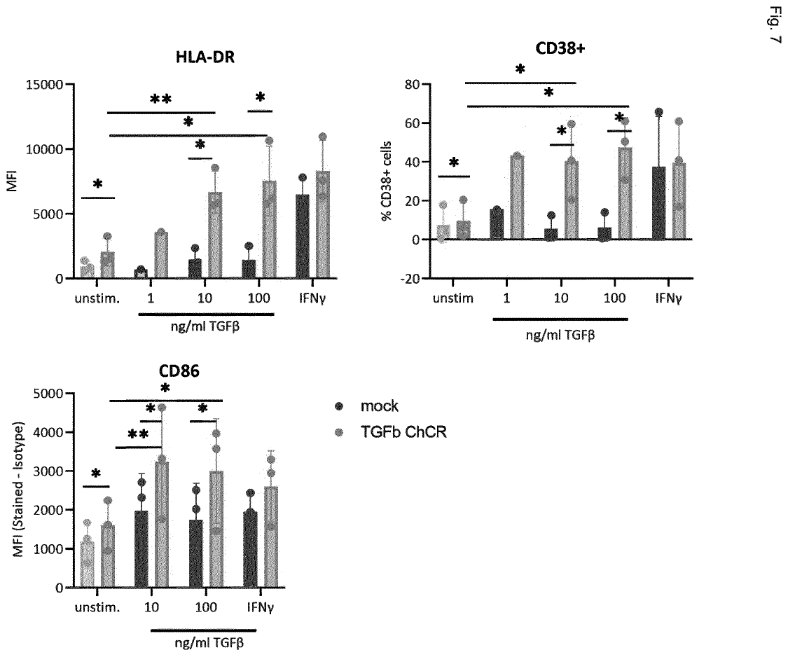

Next, the inventors transduced primary human monocytes with the TGF-β ChCR lentiviral vector and as control served monocytes transduced with a control lentivirus (“mock”) or were left untransduced. After transduction, the inventors differentiated the monocytes to macrophages. The macrophages were either stimulated with IFNγ for M1 activation, with 1, 10 or 100 ng/mL TGF-β or were left unstimulated. After stimulation, the inventors harvested the cells and analyzed the expression of HLA-DR, CD38 and Cd86 as marker for M1. (FIG. 7)

### Example 6: Stimulation of the IL-10 and the TGF-β Chimeric Cytokine Receptor

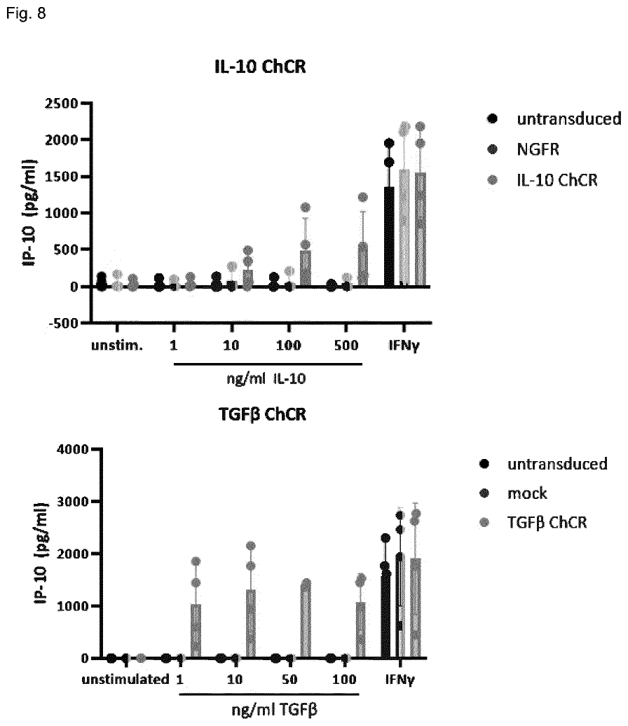

The inventors transduced primary human monocytes with the ChCR (IL-10 or TGF-β) lentiviral vector and as control served monocytes transduced with a control lentivirus (“mock”) or were left untransduced. After transduction, the inventors differentiated the monocytes to macrophages. After stimulating with 1, 10 and 100 ng/mL TGF-β in case of the TGF-β ChCR and 1, 10, 100 and 500 ng/mL IL-10 in case of the IL-10 ChCR, the secretion of IP-10 by the stimulated macrophages was measured by ELISA. As control served medium containing no cytokines (“0”) or IFN-γ. IP-10 is normally only secreted upon IFN-γ stimulation, but the inventors show in FIG. 8 that it is also secreted when the macrophages express a ChCR and are stimulated with the respective cytokine (e.g. IL-10 or TGF-β).

### Example 7: Tumoricidal Effect of Macrophage Conditioned Medium

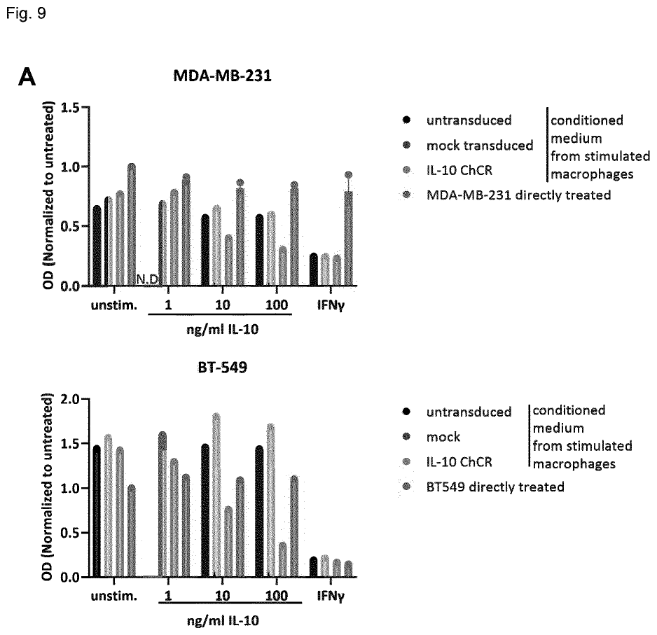

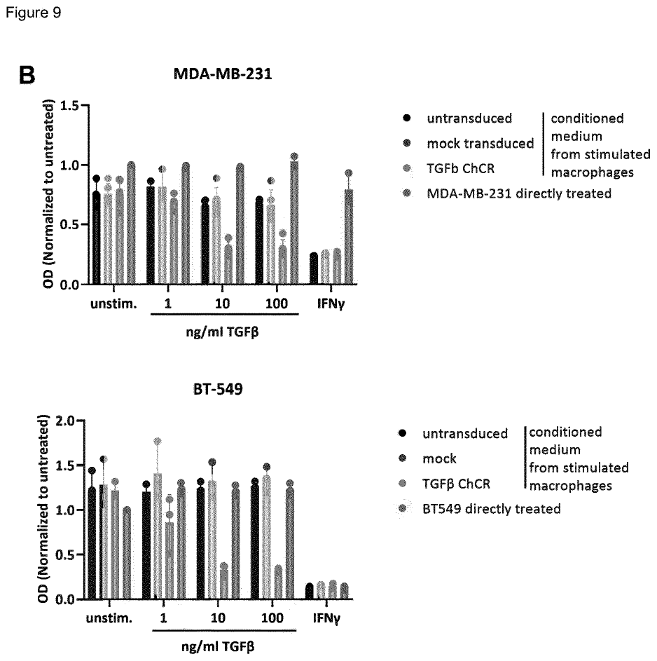

The inventors transduced primary human monocytes from healthy donors with an empty lentiviral vector (“mock”), with IL-10 ChCR (FIG. 9A), or TGFβ ChCR (FIG. 9B) expressing lentiviruses or were left untransduced. After differentiation for 7 days to monocyte derived macrophages (MDMs), they were stimulated for 2 days with medium containing no cytokine, IFN-γ or IL-10 or TGF-β as indicated in FIG. 9. The conditioned medium from the stimulated macrophages was used to treat MDA-MB-231 or BT-549 cell line for 3 days. Viability of the tumor cells was assessed using a WST1 assay. The inventors show that conditioned medium from ChCR expressing macrophages stimulated with IL-10 or TGFβ has a negative impact upon tumor cell viability similar to the conditioned medium of IFNγ stimulated macrophages (FIG. 9). To control for potential effects of recombinant IL-10 or TGF-β on MDA-MB-231 and BT-549, cell lines were directly treated with corresponding cytokines. Results show that the effect on the viability is indeed caused by factors secreted by ChCR expressing macrophages and not by recombinant cytokines.

### Example 8

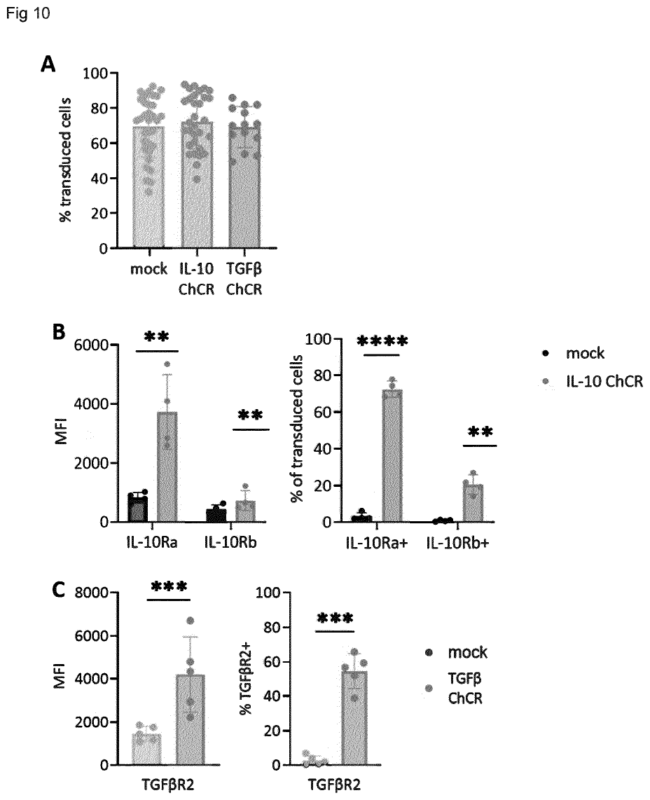

The inventors can transduce primary human macrophages with an efficacy of 69.62%±16.97%, 72.3%±15.5% and 69.3%±11.58% with mock, IL-10 ChCR and TGF-β ChCR lentiviruses, respectively (mean±SD) (FIG. 10A). They also verified the expression of the IL-10 ChCR (FIG. 10B) and of the TGF-β ChCR (FIG. 10C) on the surface of the transduced cells.

### Example 9: Sequences

TABLE 1
enlists the sequences of the invention, which also appending in the ST.26 sequence
protocol. Recurring sections of sequences are labeled with a corresponding consistent font.

| | |
|---|---|
| mCAR Sequences | SEQ ID NO: |

Car parts

| | | |
|---|---|---|
| huCD8a signal | MALPVTALLLPLALLLHAARP | 1 |
| peptide | | |
| Anti-huCD19 | DIQMTQTTSSLSASLGDRVTISCRASQDISKYLNWYQQKPDGTVKLLIYHTSRLHS | 2 |
| FCM63 VL | GVPSRFSGSGSGTDYSLTISNLEQEDIATYFCQQGNTLPYTFGGGTKLEIT | |
| Glycine-Serine | GGGGSGGGGSGGGGS | 3 |
| linker | | |
| Anti-huCD19 | EVKLQESGPGLVAPSQSLSVTCTVSGVSLPDYGVSWIRQPPRKGLEWLGVIWGS | 4 |
| FCM63 VH | ETTYYNSALKSRLTIIKDNSKQVFLKMNSLQTDDTAIYYCAKHYYYGGSYAMDY | |
| | WGQGTSVTVSS | |
| Anti-huEGFR | DILLTQSPVILSVSPGERVSFSCRASQSIGTNIHWYQQRTNGSPRRLLIKYASESISGIPS | 5 |
| Cetuximab VL | RFSGSGSGTDFTLSINSVESEDIADYYCQQNNNWPTTFGAGTKLELK | |
| Withlow Linker | GSTSGSGKPGSGEFSTKG | 6 |
| Anti-huEGFR | QVQLKQSGPGLVQPSQSLSITCTVSGFSLTNYGVHWVRQSPGKGLEWLGVIWSG | 7 |
| Cetuximab VH | GNTDYNTPFTSRLSINKDNSKSQVFFKMNSLQSNDTAIYYCARALTYYDYEFAYWG | |
| | QGTLVTVSA | |
| hUCD8a stalk | TTTPAPRPPTPAPTIASQPLSLRPEACRPAAGGAVHTRGLDFACD | 8 |
| domain | | |
| huCD8a transmembrane domain | 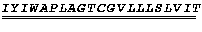 | 9 |
| huCD3ζ signaling | RVKFSRSADAPAYQQGQNQLYNELNLGRREEYDVLDKRRGRDPEMGGKPQRRKN | 10 |
| domain | PQEGLYNELQKDKMAEAYSEIGMKGERRRGKGHDGLYQGLSTATKDTYDALHMQ | |
| | ALPPR | |
| huCD32a | CRKKRISANSTDPVKAAQFEPPGRQMIAIRKRQLEETNNDYETADGGYMTLNPRA | 11 |
| signaling domain | PTDDDKNIYLTLPPNDHVNSNN | |
| huFCERG | RLKIQVRKAAITSYEKSDGVYTGLSTRNQETYETLKHEKPPQ | 12 |
| signaling domain | | |

mCAR full sequences

| | | |
|---|---|---|
| Anti-huCD19_ FCM63_CD8a_huCD3ζ | 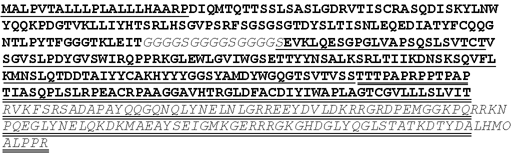 | 13 |
| Anti-huCD19_ FCM63_CD8a_ huCD32a | 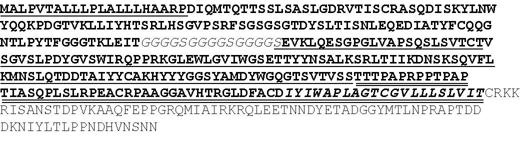 | 14 |
| Anti-huCD19_ FCM63_CD8a_ huFCERG | 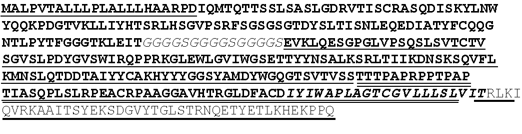 | 15 |
| Anti-huEGFR Cetuximab_ CD8a_huCD3ζ | 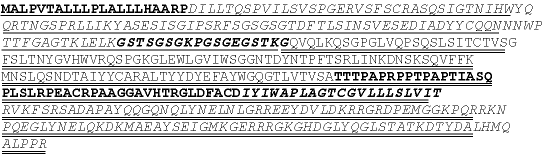 | 16 |
| Anti-huEGFR Cetuximab_ CD8a_huCD32a | 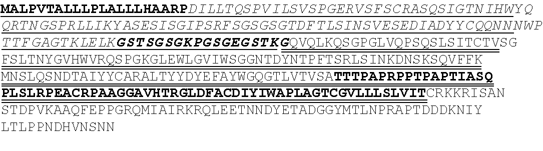 | 17 |
| Anti-huEGFR Cetuximab_ CD8a_huFCERG | 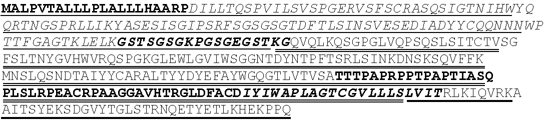 | 18 |

ChCR Sequences

| | | |
|---|---|---|
| huIFNGR1 Signal Peptide | 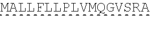 | 19 |
| huIFNGR2 Signal peptide | 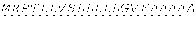 | 20 |
| huIL-10Ra Extracellular Domain | 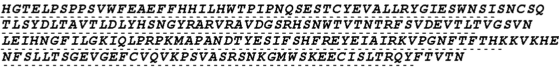 | 21 |
| huIL-10Rb Extracellular Domain | 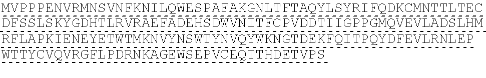 | 22 |
| huTGFβR1 Extracellular Domain | 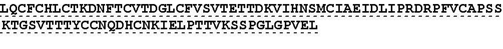 | 23 |
| huTGFβR2 Extracellular Domain | 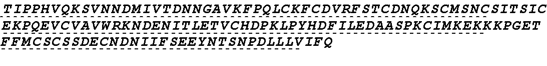 | 24 |
| Glycine-Serine linker | 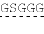 | 25 |
| huIFNGR1 Transmembrane Domain | 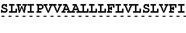 | 26 |
| huIFNGR2 Transmembrane Domain | 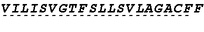 | 27 |
| huIFNGR1 Intracellular Domain | 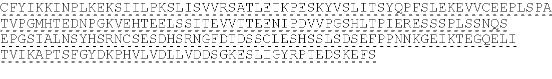 | 28 |
| huIFNGR2 Intracellular Domain | 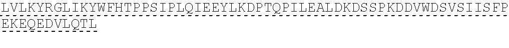 | 29 |
| Linker P2A cleavage site | 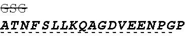 | 30 |

ChCR full sequences

| | | |
|---|---|---|
| huIL10Ra- huIFNGR1_P2A_ huIL10Rb-huIFNGR2 | 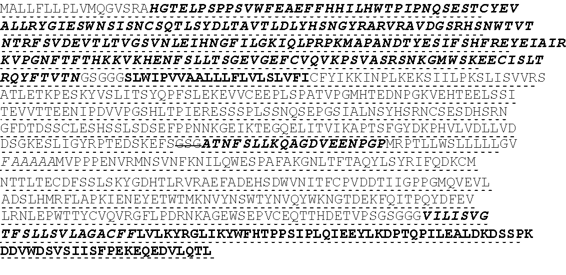 | 31 |
| huTGFβR1- IFNGR1_P2A_ huTGFβR2-IFNGR2 | 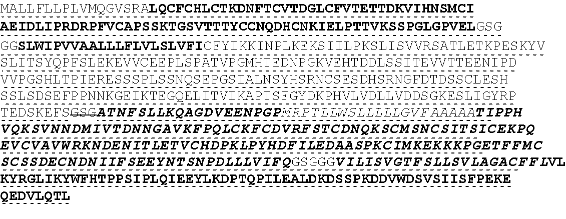 | 32 |
| IL10Ra-IFNGR1 | MALLFLLPLVMQGVSRAHGTELPSPPSVWFEAEFFHHILHWTPIPNQSESTCYEVA | 53 |
| | LLRYGIESWNSISNCSQTLSYDLTAVTLDLYHSNGYRARVRAVDGSRHSNWTVTNT | |
| | RFSVDEVTLTVGSVNLEIHNGFILGKIQPRPKMAPANDTYESIFSHFREYEIAIRKVP | |
| | GNFTFTHKKVKHENFSLLTSGEVGEFCVQVKPSVASRSNKGMWSKEECISLTRQYF | |
| | TVTNGSGGGSLWIPVVAALLLFLVLSLVFICFYIKKINPLKEKSIILPKSLISVVRSATLET | |
| | KPESKYVSLITSYQPFSLEKEVVCEEPLSPATVPGMHTEDNPGKVEHTEELSSITEVVT | |
| | TEENIPDVVPGSHLTPIERESSSPLSSNQSEPGSIALNSYHSRNCSESDHSRNGFDTD | |
| | SSCLESHSSLSDSEFPPNNKGEIKTEGQELITVIKAPTSFGYDKPHVLVDLLVDDSGKE | |
| | SLIGYRPTEDSKEFS | |
| IL10Rb-IFNGR2 | MRPTLLWSLLLLLGVFAAAAAMVPPPENVRMNSVNFKNILQWESPAFAKGNLTFT | 54 |
| | AQYLSSYRIFQDKCMNTTLTECDFSSLSKYGDHTLRVRAEFADEHSDWVNITFPVD | |
| | DTIIGPPGMQVEVLADSLHMRFLAPKIENEYETWTMKNVYNSWTYNVQYWKNG | |
| | TDEKFQITPQYDFEVLRNLEPWTTCVQVRGFLPDRNKAGEWSEPVCEQTTHDET | |
| | VPSGSGGGVILISVGTFSLLSVLAGACFFLVLKYRGLIKYWFHTPPSIPLQIEEYLKDPT | |
| | QPILEALDKDSSPKDDVWDSVSIISFPEKEQEDVLQTL | |
| TGFbR1-IFNGR1 | MALLFLLPLVMQGVSRALQCFCHLCTKDNFTCVTDGLCFVSVTETTDKVIHNSMCI | 55 |
| | AEIDLIPRDRPFVCAPSSKTGSVTTTYCCNQDHCNKIELPTTVKSSPGLGPVELGSGG | |
| | GSLWIPVVAALLLFLVLSLVFICFYIKKINPLKEKSIILPKSLISVVRSATLETKPESKYVSLI | |
| | TSYQPFSLEKEVVCEEPLSPATVPGMHTEDNPGKVEHTEELSSITEVVTTEEINIPDVV | |
| | PGSHLTPIERESSSPLSSNQSEPGSIALNSYHSRNCSESDHSRNGFDTDSSCLESHSSL | |
| | SDSEFPPNNKGEIKTEGQELITVIKAPTSFGYDKPHVLVDLLVDDSGKESLIGYRPTED | |
| | SKEFS | |
| TGFbR2-IFNGR2 | MRPTLLWSLLLLLGVFAAAAATIPPHVQKSVNNDMIVTDNNGAVKFPQLCKFCDV | 56 |
| | RFSTCDNQKSCMSNCSITSICEKPQEVCVAVWRKNDENITLETVCHDPKLPYHDFIL | |
| | EDAASPKCIMKEKKKPGETFFMCSCSSDECNDNIIFSEEYNTSNPDLLLVIFQGSGGG | |
| | VILISVGTFSLLSVLAGACFFLVLKYRGLIKYWFHTPPSIPLQIEEYLKDPTQPILEALDK | |
| | DSSPKDDVWWDSVSIISFPEKEQEDVLQTL | |
| SIRPa MiRNA Sequences | 5′-3′ orientation | |
| antisense 1 | ATGTTACCGATGCGGATGGAAA | 33 |
| antisense 2 | TAGATCCAGTGTTCTCAGCGGC | 34 |
| antisense 3 | ATTAGATCCAGTGTTCTCAGCG | 35 |
| antisense 4 | TCTTCTCGGGCTCATGCAACCT | 36 |
| antisense 5 | TTGTGTCCTGTGTTATTTCTCT | 37 |
| antisense 6 | TTCAGGTCTGCATATGTGATAT | 38 |
| antisense 7 | ACACCTCAGTTTCAAGATTGCA | 39 |
| antisense 8 | AGGCGCAGGACGCGGCGAGCAG | 40 |
| antisense 9 | TCCTTGTTCTCTGTAACGGTTG | 41 |
| antisense 10 | TCCTGACCAGGCGCAGGACGCG | 42 |
| sense 1 | TTTCCATCCGAATCGGTAACAC | 43 |
| sense 2 | GCCGCTGAGACCACTGGATCTC | 44 |
| sense 3 | CGCTGAGAACCCTGGATCTAAC | 45 |
| sense 4 | AGGTTGCATGCGCCCGAGAAGC | 46 |
| sense 5 | AGAGAAATAAAACAGGACACAC | 47 |
| sense 6 | ATATCACATACGCAGACCTGAC | 48 |
| sense 7 | TGCAATCTTGCAACTGAGGTGC | 49 |
| sense 8 | CTGCTCGCCGAGTCCTGCGCCC | 50 |
| sense 9 | CAACCGTTACCGAGAACAAGGC | 51 |
| sense 10 | CGCGTCCTGCACCTGGTCAGGC | 52 |
| IL-10Rb-IFNGR1-2a-IL- | MALLFLLPLVMQGVSRAMVPPPENVRMNSVNFKNILQWESPAFA | 57 |
| 10Ra-IFNGR2 | KGNLTFTAQYLSYRIFQDKCMNTTLTECDFSSLSKYGDHTLRVRAEF | |
| | ADEHSDWVNITFCPVDDTIIGPPGMQVEVLADSLHMRFLAPKIEN | |
| | EYETWWTMKNVYNSWTYNVQYWKNGTDEKFQITPQYDFEVLRNLE | |
| | PWTTYCVQVRGFLPDRNKAGEWSEPVCEQTTHDETVPSGSGGGS | |
| | LWIPVVAALLLFLVLSLVFICFYIKKINPLKEKSIILPKSLISVVRSATLET | |
| | KPESKYVSLITSYQPFSLEKEVVCEEPLSPATVPGMHTEDNPGKVEH | |
| | TEELSSITEVVTTEENIPDVVPGSHLTPIERESSSPLSSNQSEPGSIALN | |
| | SYHSRNCSESDHSRNGFDTDSSCLESHSSLSDSEFPPNNKGEIKTEG | |
| | QELITVIKAPTSFGYDKPHVLVDLLVDDSGKESLIGYRPTEDSKEFSG | |
| | SGATNFSLLKQAGDVEENPGPMRPTLLWSLLLLLGVFAAAAAHGT | |
| | ELPSPPSVWFEAEFFHHILHWTPIPNQSESTCYEVALLRYGIESWNSI | |
| | SNCSQTLSYDLTAVTLDLYHSNGYRARVRAVDGSRHSNWTVTNTR | |
| | FSVDEVTLTVGSVNLEIHNGFILGKIQLPRPKMAPANDTYESIFSHFR | |
| | EYEIAIRKVPGNFTFTHKKVKHENFSLLTSGEVGEFCVQVKPSVASRS | |
| | NKGMWSKEECISLTRQYFTVTNGSGGGVILISVGTFSLLSVLAGACF | |
| | FLVLKYRGLIKYWFHTPPSIPLQIEEYLKDPTQPILEALDKDSSPKDDV | |
| | WDSVSIISFPEKEQEDVLQTL | |
| TGFbR1-IFNGR2-2a- | MALLFLLPLVMQGVSRALQCFCHLCTKDNFTCVTDGLCFVSVTETT | 58 |
| TGFbR2-IFNGR1 | DKVIHNSMCIAEIDLIPDRPFVCAPSSKTGSVTTTYCCNQDHCNKI | |
| | ELPTTVKSSPGLGPVELGSGGGVILISVGTFSLLSVLAGACFFLVLKYR | |
| | GLIKYWFHTPPSIPLQIEEYLKDPTQPILEALDKDSSPKDDVWDSVSII | |
| | SFPEKEQEDVLQTLGSGATNFSLLKQAPGVEENPGPMRPTLLWSLL | |
| | LLLGVFAAAAATIPPHVQKSVNNDMIVTDNNGAVKFPQLCKFCDV | |
| | RFSTCDNQKSCMSNCSITSICEKPQEVCVAVWRKNDENITLETVCH | |
| | DPKLPYHDFILEDAASPKCIMKEKKKPGETFFMCSCSSDECNDNIIFS | |
| | EEYNTSNPDLLLVIFQGSGGGSLWIPVVAALLLFLVLSLVFICFYIKKI | |
| | NPLKEKSIILPKSLISVVRSATLETKPESKYVSLITSYQPFSLEKEVVCEE | |
| | PLSPATVPGMHTEDNPGKVEHTEELSSITEVVTTEENIPDVVPGSHL | |
| | TPIERESSSPLSSNQSEPGSIALNSYHSRNCSESDHSRNGFDTDSSCL | |
| | ESHSSLSDSEFPPNNKGEIKTEGQELITVIKAPTSFGYDKPHVLVDLL | |
| | VDDSGKESLIGYRPTEDSKEFS | |
| IL-10Rb-IFNGR1 | MALLFLLPLVMQGVSRAMVPPPENVRMNSVNFKNILQWESPAFA | 59 |
| | KGNLTFTAQYLSYRIFQDKCMNTTLTECDFSSLSKYGDHTLRVRAEF | |
| | ADEHSDWVNITFCPVDDTIIGPPGMQVEVLADSLHMRFLAPKIEN | |
| | EYETWTMKNVYNSWTYNVQYWKNGTDEKFQITQPQYDFEVLRNLE | |
| | PWTTYCVQVRGFLPDRNKAGEWSEPVCEQTTHDETVPSGSGGGS | |
| | LWIPVVAALLLFLVLSLVFICFYIKKINPLKEKSIILPKSLISVVRSATLET | |
| | KPESKYVSLITSYQPFSLEKEVVCEEPLSPATVPGMHTEDNPGKVEH | |
| | TEELSSITEVVTTEENIPDVVPGSHLTPIERESSSPLSSNQSEPGSIALN | |
| | SYHSRNCSESDHSRNGFDTDSSCLESHSSLSDSEFPPNNKGEIKTEG | |
| | QELITVIKAPTSFGYDKPHVLVDLLVDDSGKESLIGYRPTEDSKEFS | |
| IL-10Ra-IFNGR2 | MRPTLLWSLLLLLGVFAAAAAHGTELPSPPSVWFEAEFFHHILHWT | 60 |
| | PIPNQSESTCYEVALLRYGIESWNSISNCSQTLSYDLTAVTLDLYHSN | |
| | GYRARVRAVDGSRHSNWTVTNTRFSVDEVTLTVGSVNLEIHNGFIL | |
| | GKIQLPRPKMAPANDTYESIFSHFREYEIAIRKVPGNFTFTHKKVKH | |
| | ENFSLLTSGEVGEFCVQVKPSVASRSNKGMWSKEECISLTRQYFTV | |
| | TNGSGGGVILISVGTFSLLSVLAGACFFLVLKYRGLIKYWFHTPPSIPL | |
| | QIEEYLKDPTQPILEALDKDSSPKDDVWDSVSIISFPEKEQEDVLQTL | |
| TGFbR1-IFNGR2 | MALLFLLPLVMQGVSRALQCFCHLCTKDNFTCVTDGLCFVSVTETT | 61 |
| | DKVIHNSMCIAEIDLIPRDRPFVCAPSSKTGSVTTTYCCNQDHCNKI | |
| | ELPTTVKSSPGLGPVELGSGGGVILISVGTFSLLSVLGACFFLVLKYR | |
| | GLIKYWFHTPPSIPLQIEEYLKDPTQPILEALDKDSSPKDDVWDSVSII | |
| | SFPEKEQEDVLQTL | |
| TGFbR2-IFNGR1 | MRPTLLWSLLLLLGVFAAAAATIPPHVQKSVNNDMIVTDNNGAVK | 62 |
| | FPQLCKFCDVRFSTCDNQKSCMSNCSITSICEKPQEVCVAVWRKN | |
| | DENITLETVCHDPKLPYHDFILEDAASPKCIMKEKKKPGETFFMCSC | |
| | SSDECNDNIIFSEEYNTSNPDLLLVIFQGSGGGSLWIPVVALLLFLV | |
| | LSLVFICFYIKKINPLKEKSIILPKSLISVVRSATLETKPESKYVSLITSYQP | |
| | FSLEKEVVCEEPLSPATVPGMHTEDNPGKVEHTEELSSITEVVTTEE | |
| | NIPDVVPGSHLTPIERESSSPLSSNQSEPGSIALNSYHSRNCSESDHS | |
| | RNGFDTDSSCLESHSSLSDSEFPPNNKGEIKTEGQELITVIKAPTSFG | |
| | YDKPHVLVDLLVDDSGKESLIGYRPTEDSKEFS | |

1. An isolated monocyte or macrophage comprising a chimeric cytokine receptor (ChCR) polypeptide heterodimer comprising or consisting of a first ChCR polypeptide and a second ChCR polypeptide;
wherein the first ChCR polypeptide comprises:
a first extracellular domain,
a first type 1 transmembrane domain,
optionally, a first flexible linker domain, linking said first extracellular domain and said first transmembrane domain,
a first intracellular domain,
and wherein the second ChCR polypeptide comprises:
a second extracellular domain,
a second type 1 transmembrane domain,
optionally, a second flexible linker domain, linking said second extracellular domain and said second transmembrane domain,
a second intracellular domain,
wherein
the first extracellular domain and the second extracellular domain are able to induce dimerization of said ChCR polypeptide heterodimer upon binding to a cytokine selected from IL-10 and TGFβ; and
the first intracellular domain and the second intracellular domain are able to activate IFNγ receptor/Jak1/Jak2/STAT1 signalling in monocytes and/or macrophages upon dimerization of said ChCR polypeptide heterodimer.

2. The isolated monocyte or macrophage according to claim 1, wherein
the first extracellular domain is or comprises an IL-10 receptor α (CDW210A) extracellular domain;
the second extracellular domain is or comprises an IL-10 receptor β (CDW210B) extracellular domain;
the first intracellular domain is or comprises an IFNγ receptor 1 (CD119) intracellular domain; and
the second intracellular domain is or comprises an IFNγ receptor 2 intracellular domain.

3. The isolated monocyte or macrophage according to claim 1 or 2, wherein said ChCR polypeptide heterodimer comprises:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 21,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 22,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
wherein said ChCR polypeptide heterodimer has a biological activity of 85%, particularly ≥90%, ≥95% of a ChCR heterodimer of the sequences SEQ ID NO 53 and SEQ ID NO 54.

4. The isolated monocyte or macrophage according to claim 1, wherein
the first extracellular domain is or comprises an IL-10 receptor α (CDW210A) extracellular domain;
the second extracellular domain is or comprises an IL-10 receptor β (CDW210B) extracellular domain;
the first intracellular domain is or comprises an IFNγ receptor 2 intracellular domain; and
the second intracellular domain is or comprises an IFNγ receptor 1 (CD119) intracellular domain.

5. The isolated monocyte or macrophage according to claim 1 or 4, wherein said ChCR polypeptide heterodimer comprises:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 21,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 22,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
wherein said ChCR polypeptide heterodimer has a biological activity of 85%, particularly ≥90%, ≥95% of a ChCR heterodimer of the sequences SEQ ID NO 59 and SEQ ID NO 60.

6. The isolated monocyte or macrophage according to claim 1, wherein
the first extracellular domain is or comprises a TGFβ receptor type I extracellular domain;
the second extracellular domain is or comprises a TGFβ receptor type II extracellular domain;
the first intracellular domain is or comprises an IFNγ receptor 1 (CD119) intracellular domain; and
the second intracellular domain is or comprises an IFNγ receptor 2 intracellular domain.

7. The isolated monocyte or macrophage according to claim 1 or 6, wherein said ChCR polypeptide heterodimer comprises:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 23,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 24,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
wherein said ChCR polypeptide heterodimer has a biological activity of 85%, particularly ≥90%, ≥95% of a ChCR heterodimer of the sequences SEQ ID NO 55 and SEQ ID NO 56.

8. The isolated monocyte or macrophage according to claim 1, wherein
the first extracellular domain is or comprises a TGFβ receptor type I extracellular domain;
the second extracellular domain is or comprises a TGFβ receptor type II extracellular domain;
the first intracellular domain is or comprises an IFNγ receptor 2 intracellular domain; and
the second intracellular domain is or comprises an IFNγ receptor 1 (CD119) intracellular domain.

9. The isolated monocyte or macrophage according to claim 1 or 8, wherein said ChCR polypeptide heterodimer comprises:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 23,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 24,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
wherein said ChCR polypeptide heterodimer has a biological activity of 85%, particularly ≥90%, ≥95% of a ChCR heterodimer of the sequences SEQ ID NO 61 and SEQ ID NO 62.

10. An isolated monocyte or macrophage comprising a nucleic acid molecule encoding the ChCR polypeptide as described in any one of claims 1 to 9.

11. The isolated monocyte or macrophage according to claim 10, wherein the nucleic acid molecule is comprised in an expression vector, wherein the ChCR polypeptide heterodimer is under control of a promoter sequence operable in a mammalian monocyte or macrophage,
particularly wherein the expression vector is selected from the group of a viral vector, a plasmid, a DNA molecule, or an RNA molecule,
more particularly wherein the expression vector is a lentiviral vector.

12. The isolated monocyte or macrophage according to any one of the preceding claims, wherein the monocyte or macrophage additionally comprises an inhibitory nucleic acid molecule directed against SIRβα.

13. The isolated monocyte or macrophage according to claim 12, wherein the inhibitory nucleic acid molecule directed against SIRβα comprises a sequence selected from the group of SEQ ID NO 33 to SEQ ID NO 52.

14. The isolated monocyte or macrophage according to any one of claims, wherein the monocyte or macrophage additionally comprises a chimeric antigen receptor (CAR) polypeptide, comprising:
an extracellular antigen binding domain, in particular a Fab-fragment,
a transmembrane domain, in particular a CD8 transmembrane domain, and
an intracellular domain comprising at least one ITAM motif, in particular the intracellular domain of a FC-receptor, more particular the intracellular domain of a FCgamma-receptor or of CD32a,
optionally, a linker domain, linking said extracellular antigen binding domain and said transmembrane domain.

15. The isolated monocyte or macrophage according to claim 14, wherein the CAR polypeptide comprises a sequence having at least ≥80%, ≥85%, ≥90%, ≥92%, ≥94%, ≥95%, ≥96%, ≥97%, ≥98%, ≥99%, or 100% identity to any one of SEQ ID NO 13 to SEQ ID NO 18.

16. An isolated monocyte or macrophage according to any one of the preceding claims for use in treatment or prevention of cancer.

17. A kit comprising
an expression vector encoding a ChCR polypeptide as described in any one of claims 1 to 9;
an inhibitory nucleic acid molecule directed against SIRβα as described in any one of claim 12 or 13; and
an expression vector encoding a CAR polypeptide as described in any one of claim 14 or 15.

18. A method for monocyte modification, comprising:
i. providing a monocyte obtained from a mammalian donor,
ii. inserting into said monocyte a nucleic acid sequence encoding a ChCR polypeptide as described in any one of claims 1 to 9;
iii. maintaining said monocyte under cell culture conditions.

19. The method according to claim 18, wherein additionally,
an inhibitory nucleic acid molecule directed against SIRβα as described in any one of claim 12 or 13; and/or
a CAR polypeptide as described in any one of claim 14 or 15;
is inserted into said monocyte.

20. A ChCR polypeptide heterodimer comprising:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 21,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 22,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29.

21. A ChCR polypeptide heterodimer comprising:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 21,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 22,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28.

22. A ChCR polypeptide heterodimer comprising:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 23,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 24,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29.

23. A ChCR polypeptide heterodimer comprising:
a first extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 23,
a first transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 27, and
a first intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 29;
a second extracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 24,
a second transmembrane domain comprising a sequence having at least ≥80%, ≥85%, ≥90%, ≥92%, ≥94%, ≥95%, ≥96%, ≥97%, ≥98%, ≥99%, or 100% identity to SEQ ID NO 26, and
a second intracellular domain comprising a sequence having at least ≥80%, ≥85%, ≥90%, ≥92%, ≥94%, ≥95%, ≥96%, ≥97%, ≥98%, ≥99%, or 100% identity to SEQ ID NO 28.


SEQUENCE LISTING (62 sequences)

SEQ ID NO: 1 - protein, 21 aa, synthetic construct
MALPVTALLLPLALLLHAARP

SEQ ID NO: 2 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 3 - protein, 15 aa, synthetic construct
GGGGSGGGGSGGGGS

SEQ ID NO: 4 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 5 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 6 - protein, 18 aa, synthetic construct
GSTSGSGKPGSGEGSTKG

SEQ ID NO: 7 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 8 - protein, 45 aa, synthetic construct
TTTPAPRPPTPAPTIASQPLSLRPEACRPAAGGAVHTRGLDFACD

SEQ ID NO: 9 - protein, 21 aa, synthetic construct
IYIWAPLAGTCGVLLLSLVIT

SEQ ID NO: 10 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 11 - protein, 77 aa, synthetic construct
CRKKRISANSTDPVKAAQFEPPGRQMIAIRKRQLEETNNDYETADGGYMTLNPRAPTDDDKNIYLTLPPNDHVNSNN

SEQ ID NO: 12 - protein, 42 aa, synthetic construct
RLKIQVRKAAITSYEKSDGVYTGLSTRNQETYETLKHEKPPQ

SEQ ID NO: 13 - protein, 442 aa, synthetic construct
  REGION 1..21: note = signal peptide
  REGION 22..128: note = VL
  REGION 129..143: note = linker
  REGION 144..263: note = VH
  REGION 264..308: note = huCD8a stalk domain
  REGION 330..442: note = huCD3zeta
  REGION 309..329: note = huCD8a transmembrane domain
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 14 - protein, 406 aa, synthetic construct
  REGION 1..21: note = signal peptide
  REGION 22..128: note = VL
  REGION 129..143: note = linker
  REGION 144..263: note = VH
  REGION 264..308: note = stalk domain
  REGION 309..329: note = transmembrane domain
  REGION 330..406: note = huCD32a signaling domain
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITCRKKRISANSTDPVKAAQFEPPGRQMIAIRKRQLEETNNDYETADGGYMTLNPRAPTDDDKNIYLTLPPNDHVNSNN

SEQ ID NO: 15 - protein, 371 aa, synthetic construct
  REGION 1..21: note = signal peptide
  REGION 22..128: note = VL
  REGION 129..143: note = linker
  REGION 144..263: note = VH
  REGION 265..308: note = huCD8a stalk domain
  REGION 309..329: note = huCD8a transmembrane domain
  REGION 330..371: note = huFCERG signaling domain
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITRLKIQVRKAAITSYEKSDGVYTGLSTRNQETYETLKHEKPPQ

SEQ ID NO: 16 - protein, 444 aa, synthetic construct
  REGION 1..21: note = signal peptide
  REGION 22..128: note = VL
  REGION 129..146: note = Withlow Linker
  REGION 147..265: note = Anti-huEGFR Cetuximab VH
  REGION 266..310: note = huCD8a stalk domain
  REGION 311..331: note = huCD8a transmembrane domain
  REGION 332..444: note = huCD3zeta signaling domain
MALPVTALLLPLALLLHAARPDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGSTSGSGKPGSGEGSTKGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSATTTPAPRPPTPAPTIASQPLSLRPEACRPAAGGAVHTRGLDFACDIYIWAPLAGTCGVLLLSLVITRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 408 aa, synthetic construct
  REGION 1..21: note = signal peptide
  REGION 22..128: note = Anti-huEGFR Cetuximab VL
  REGION 129..146: note = Withlow Linker
  REGION 147..265: note = Anti-huEGFR Cetuximab VH
  REGION 266..310: note = huCD8a stalk domain
  REGION 311..331: note = huCD8a transmembrane domain
  REGION 332..408: note = huCD32a signaling domain
MALPVTALLLPLALLLHAARPDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGSTSGSGKPGSGEGSTKGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSATTTPAPRPPTPAPTIASQPLSLRPEACRPAAGGAVHTRGLDFACDIYIWAPLAGTCGVLLLSLVITCRKKRISANSTDPVKAAQFEPPGRQMIAIRKRQLEETNNDYETADGGYMTLNPRAPTDDDKNIYLTLPPNDHVNSNN

SEQ ID NO: 18 - protein, 373 aa, synthetic construct
  REGION 1..21: note = signal peptide
  REGION 22..128: note = Anti-huEGFR Cetuximab VL
  REGION 129..146: note = Withlow Linker
  REGION 147..265: note = Anti-huEGFR Cetuximab VH
  REGION 266..310: note = huCD8a stalk domain
  REGION 311..331: note = huCD8a transmembrane domain
  REGION 332..373: note = huFCERG signaling domain
MALPVTALLLPLALLLHAARPDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGSTSGSGKPGSGEGSTKGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSATTTPAPRPPTPAPTIASQPLSLRPEACRPAAGGAVHTRGLDFACDIYIWAPLAGTCGVLLLSLVITRLKIQVRKAAITSYEKSDGVYTGLSTRNQETYETLKHEKPPQ

SEQ ID NO: 19 - protein, 17 aa, synthetic construct
MALLFLLPLVMQGVSRA

SEQ ID NO: 20 - protein, 21 aa, synthetic construct
MRPTLLWSLLLLLGVFAAAAA

SEQ ID NO: 21 - protein, 214 aa, synthetic construct
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 22 - protein, 201 aa, synthetic construct
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPS

SEQ ID NO: 23 - protein, 93 aa, synthetic construct
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 24 - protein, 144 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 25 - protein, 5 aa, synthetic construct
GSGGG

SEQ ID NO: 26 - protein, 21 aa, synthetic construct
SLWIPVVAALLLFLVLSLVFI

SEQ ID NO: 27 - protein, 21 aa, synthetic construct
VILISVGTFSLLSVLAGACFF

SEQ ID NO: 28 - protein, 223 aa, synthetic construct
CFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 29 - protein, 69 aa, synthetic construct
LVLKYRGLIKYWFHTPPSIPLQIEEYLKDPTQPILEALDKDSSPKDDVWDSVSIISFPEKEQEDVLQTL

SEQ ID NO: 30 - protein, 19 aa, synthetic construct
ATNFSLLKQAGDVEENPGP

SEQ ID NO: 31 - protein, 819 aa, synthetic construct
  REGION 1..17: note = signal peptide
  REGION 18..231: note = huIL-10Ra Extracellular Domain
  REGION 232..236: note = linker
  REGION 237..257: note = huIFNGR1 Transmembrane Domain
  REGION 258..480: note = huIFNGR1 Intracellular Domain
  REGION 481..483: note = linker
  REGION 484..502: note = P2A cleavage site
  REGION 503..523: note = huIFNGR1 Transmembrane Domain
  REGION 524..724: note = huIL-10Rb Extracellular Domain
  REGION 725..729: note = linker
  REGION 730..750: note = huIFNGR2 Transmembrane Domain
  REGION 751..819: note = huIFNGR2 Intracellular Domain
MALLFLLPLVMQGVSRAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNGSGGGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFSGSGATNFSLLKQAGDVEENPGPMRPTLLWSLLLLLGVFAAAAAMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSGSGGGVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 32 - protein, 641 aa, synthetic construct
  REGION 1..17: note = signal peptide
  REGION 18..110: note = huTGFbetaR1 Extracellular Domain
  REGION 111..115: note = linker
  REGION 116..359: note = huIFNGR1 Intracellular Domain
  REGION 360..362: note = linker
  REGION 363..381: note = P2A cleavage site
  REGION 382..402: note = huIFNGR2 Signal peptide
  REGION 403..546: note = huTGFbetaR2 Extracellular Domain
  REGION 547..551: note = linker
  REGION 552..572: note = huIFNGR2 Transmembrane Domain
  REGION 573..641: note = huIFNGR2 Intracellular Domain
MALLFLLPLVMQGVSRALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELGSGGGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFSGSGATNFSLLKQAGDVEENPGPMRPTLLWSLLLLLGVFAAAAATIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQGSGGGVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 33 - RNA, 22 nt, synthetic construct
  source 1..22: mol_type = other RNA
atgttaccgatgcggatggaaa

SEQ ID NO: 34 - RNA, 22 nt, synthetic construct
  source 1..22: mol_type = other RNA
tagatccagtgttctcagcggc

SEQ ID NO: 35 - RNA, 22 nt, synthetic construct
  source 1..22: mol_type = other RNA
attagatccagtgttctcagcg

SEQ ID NO: 36 - RNA, 22 nt, synthetic construct
  source 1..22: mol_type = other RNA
tcttctcgggctcatgcaacct

SEQ ID NO: 37 - RNA, 22 nt, synthetic construct
  source 1..22: mol_type = other RNA
ttgtgtcctgtgttatttctct

SEQ ID NO: 38 - RNA, 22 nt, synthetic construct
  source 1..22: mol_type = other RNA
ttcaggtctgcatatgtgatat

SEQ ID NO: 39 - RNA, 22 nt, synthetic construct
  source 1..22: mol_type = other RNA
acacctcagtttcaagattgca

SEQ ID NO: 40 - RNA, 22 nt, synthetic construct
  source 1..22: mol_type = other RNA
aggcgcaggacgcggcgagcag

SEQ ID NO: 41 - RNA, 22 nt, synthetic construct
  source 1..22: mol_type = other RNA
tccttgttctctgtaacggttg

SEQ ID NO: 42 - RNA, 22 nt, synthetic construct
  source 1..22: mol_type = other RNA
tcctgaccaggcgcaggacgcg

SEQ ID NO: 43 - RNA, 22 nt, synthetic construct
  source 1..22: mol_type = other RNA
tttccatccgaatcggtaacac

SEQ ID NO: 44 - RNA, 22 nt, synthetic construct
  source 1..22: mol_type = other RNA
gccgctgagaccactggatctc

SEQ ID NO: 45 - RNA, 22 nt, synthetic construct
  source 1..22: mol_type = other RNA
cgctgagaaccctggatctaac

SEQ ID NO: 46 - RNA, 22 nt, synthetic construct
  source 1..22: mol_type = other RNA
aggttgcatgcgcccgagaagc

SEQ ID NO: 47 - RNA, 22 nt, synthetic construct
  source 1..22: mol_type = other RNA
agagaaataaaacaggacacac

SEQ ID NO: 48 - RNA, 22 nt, synthetic construct
  source 1..22: mol_type = other RNA
atatcacatacgcagacctgac

SEQ ID NO: 49 - RNA, 22 nt, synthetic construct
  source 1..22: mol_type = other RNA
tgcaatcttgcaactgaggtgc

SEQ ID NO: 50 - RNA, 22 nt, synthetic construct
  source 1..22: mol_type = other RNA
ctgctcgccgagtcctgcgccc

SEQ ID NO: 51 - RNA, 22 nt, synthetic construct
  source 1..22: mol_type = other RNA
caaccgttaccgagaacaaggc

SEQ ID NO: 52 - RNA, 22 nt, synthetic construct
  source 1..22: mol_type = other RNA
cgcgtcctgcacctggtcaggc

SEQ ID NO: 53 - protein, 480 aa, synthetic construct
MALLFLLPLVMQGVSRAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNGSGGGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 54 - protein, 317 aa, synthetic construct
MRPTLLWSLLLLLGVFAAAAAMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSGSGGGVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 55 - protein, 359 aa, synthetic construct
MALLFLLPLVMQGVSRALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELGSGGGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 56 - protein, 260 aa, synthetic construct
MRPTLLWSLLLLLGVFAAAAATIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQGSGGGVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 57 - protein, 819 aa, synthetic construct
MALLFLLPLVMQGVSRAMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSGSGGGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFSGSGATNFSLLKQAGDVEENPGPMRPTLLWSLLLLLGVFAAAAAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNGSGGGVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 58 - protein, 641 aa, synthetic construct
MALLFLLPLVMQGVSRALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELGSGGGVILISVGTFSLLSVLAGACFFLVLKYRGLIKYWFHTPPSIPLQIEEYLKDPTQPILEALDKDSSPKDDVWDSVSIISFPEKEQEDVLQTLGSGATNFSLLKQAGDVEENPGPMRPTLLWSLLLLLGVFAAAAATIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQGSGGGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 59 - protein, 467 aa, synthetic construct
MALLFLLPLVMQGVSRAMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSGSGGGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 60 - protein, 330 aa, synthetic construct
MRPTLLWSLLLLLGVFAAAAAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNGSGGGVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 61 - protein, 205 aa, synthetic construct
MALLFLLPLVMQGVSRALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELGSGGGVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 62 - protein, 414 aa, synthetic construct
MRPTLLWSLLLLLGVFAAAAATIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQGSGGGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS